(19) United States
(12) Patent Application Publication
(10) Pub. No.: US 2023/0407297 A1
(43) Pub. Date: Dec. 21, 2023

(54) BIOENGINEERED WNT5A THERAPEUTICS FOR ADVANCED CANCERS

(71) Applicant: The Regents of the University of California, Oakland, CA (US)

(72) Inventors: Allen C. Gao, Oakland, CA (US); Aiming Yu, Oakland, CA (US)

(73) Assignees: The Regents of the University of California, Oakland, CA (US); The United States Government as represented by the Department of Veterans Affairs, Washington, DC (US)

(21) Appl. No.: 18/034,964

(22) PCT Filed: Nov. 3, 2021
(86) PCT No.: PCT/US2021/057939

§ 371 Date: May 2, 2023

Related U.S. Application Data
(60) Provisional application No. 63/109,292, filed on Nov. 3, 2020.

(51) Int. Cl. C12N 15/113; A61P 35/00

(52) U.S. Cl. CPC C12N 15/113; A61P 35/00; C12N 2310/141; C12N 2310/3519; C12N 2320/31

(57) ABSTRACT

The present disclosure provides methods and compositions for inhibiting Wnt5a expression in cells such as prostate cancer cells.

### CROSS REFERENCE TO RELATED APPLICATIONS

The present application claims priority to U.S. Provisional Pat. Appl. No. 63/109,292, filed on Nov. 3, 2020, which application is incorporated herein by reference in its entirety.

### BACKGROUND

Wnt signaling includes canonical ((β-catenin-dependent) and noncanonical ((β-catenin-independent) pathways. Noncanonical Wnt signaling is activated by a subset of Wnt ligands (such as Wnt5A and Wnt7b) and controls several downstream pathways, such as Ca²⁺/calmodulin-dependent protein kinase II, G proteins, Rho GTPases, or c-Jun N-terminal kinase (JNK), which are critical for cell survival, proliferation, and motility. Wnt5A plays important roles in cell proliferation, differentiation, migration, adhesion, and polarity, which plays vital roles in promoting cancer cell progression and resistance to therapies.

Several strategies have been explored for targeting Wnt signaling in cancer. However, none of them can directly target noncanonical Wnt/Wnt5A signaling. The development of new classes of therapeutics such as small interfering RNAs (siRNA) is a specific way with less off-target effects during treatment. However, unfavorable pharmacokinetic properties and side effects are major drawbacks for siRNAs to be able to advance into in vivo and clinical trials. Accordingly, the development of novel strategies targeting Wnt5A to treat cancer and overcome resistance is an urgent need. The present disclosure satisfies this need and provides other advantages as well.

### BRIEF SUMMARY

In one aspect, the present disclosure provides a tRNA-pre-miRNA chimera for inhibiting the expression of Wnt5a in a cell, the chimera comprising (i) a tRNA component comprising a first tRNA sequence at the 5′ terminus of the tRNA-pre-miRNA chimera, and a second tRNA sequence at the 3′ terminus of the tRNA-pre-miRNA chimera, wherein the first and second tRNA sequences hybridize to one another to form a tRNA structure; and (ii) a pre-miRNA sequence, located between the first and second tRNA sequences on the tRNA-pre-miRNA chimera, wherein the pre-miRNA sequence comprises an inserted heterologous Wnt5a-inhibiting RNA sequence.

In some embodiments, the heterologous Wnt5a-inhibiting RNA sequence is an siRNA or mature microRNA (mi-RNA). In some embodiments, the pre-miRNA sequence is derived from miRNA-34a. In some embodiments, the pre-miRNA sequence is derived from a mammalian pre-miRNA. In some embodiments, the mammalian pre-miRNA is a human pre-miRNA. In some embodiments, the first and/or second tRNA sequences are derived from a mammalian tRNA. In some embodiments, the mammalian tRNA is a human tRNA. In some embodiments, the first and/or second tRNA sequences are derived from a tRNA coding for an amino acid selected from the group consisting of serine, leucine, glycine, glutamate, aspartate, glutamine, arginine, cysteine, lysine, methionine, asparagine, alanine, histidine, isoleucine, phenylalanine, proline, tryptophan, tyrosine, threonine, and valine. In some embodiments, the first and/or second tRNA sequences are derived from a tRNA coding for leucine.

In some embodiments, the pre-miRNA sequence comprises (a) a first pre-miRNA-34a sequence; (b) a Wnt5a miRNA or siRNA sequence; (c) a second pre-miRNA-34a sequence; (d) a complementary Wnt5a miRNA or siRNA sequence; and (e) a third pre-miRNA-34a sequence; wherein the first and third pre-miRNA-34a sequences hybridize to one another to form a pre-miRNA structure adjacent to the tRNA structure; wherein the Wnt5a miRNA or siRNA sequence and the complementary Wnt5a miRNA or siRNA sequence hybridize to one another to form a double-stranded RNA segment adjacent to the pre-miRNA structure, on the opposite side of the pre-miRNA structure as the tRNA structure; and wherein the second pre-miRNA-34a sequence forms a stem-loop structure adjacent to the double-stranded RNA segment, on the opposite side of the double-stranded RNA segment as the pre-miRNA structure.

In some embodiments, the heterologous Wnt5a-inhibiting RNA sequence is inserted at, abutted with, or operably linked to a dicer or RNAse cleavage site within the pre-miRNA sequence. In some embodiments, the first tRNA sequence comprises the sequence shown as SEQ ID NO:9 or SEQ ID NO:10. In some embodiments, the second tRNA sequence comprises the sequence shown as SEQ ID NO:11 or SEQ ID NO:12. In some embodiments, the first pre-miRNA-34a sequence comprises the sequence shown as SEQ ID NO:13 or SEQ ID NO:14. In some embodiments, the second pre-miRNA-34a sequence comprises the sequence shown as SEQ ID NO:15, SEQ ID NO:16, or SEQ ID NO:17. In some embodiments, the third pre-miRNA-34a sequence comprises the sequence shown as SEQ ID NO:18 or SEQ ID NO:19. In some embodiments, the Wnt5a siRNA sequence comprises the sequence shown as SEQ ID NO:3, SEQ ID NO:4, SEQ ID NO:5 , or SEQ ID NO:6. In some embodiments, the complementary Wnt5a siRNA sequence comprises the sequence shown as SEQ ID NO:7 or SEQ ID NO:8. In some embodiments, the tRNA-pre-miRNA chimera comprises the sequence shown as SEQ ID NO:20 or SEQ ID NO:21. In some embodiments, the tRNA-pre-miRNA chimera comprises the sequence shown as SEQ ID NO:22, wherein (N1) corresponds to the Wnt5a siRNA or miRNA sequence, of length n, and (N2) corresponds to the complementary Wnt5a siRNA or miRNA sequence, also of length n.

In some embodiments, the introduction of the chimera into a mammalian cell results in the processing of the chimera and release of the heterologous Wnt5a-inhibiting RNA sequence in the cell. In some embodiments, the mammalian cell expresses Wnt5a, and wherein the introduction of the chimera into the cell leads to a reduction in Wnt5a expression in the cell. In some embodiments, the mammalian cell is a human cell. In some embodiments, the mammalian cell is a cancer cell. In some embodiments, the cancer cell is a prostate cancer cell. In some embodiments, the introduction of the chimera into the cancer cell inhibits the growth of the cell. In some embodiments, the cancer cell is resistant to an antiandrogen, and wherein the introduction of the tRNA-pre-miRNA chimera into the cell sensitizes the cell to the antiandrogen. In some embodiments, the tRNA-pre-miRNA chimera and the antiandrogen act synergistically to inhibit the growth of the cancer cell. In some embodiments, the co-efficient drug interaction (CDI) of the tRNA-pre-miRNA chimera and the antiandrogen is less than about 0.90, 0.85, 0.80, 0.75, or 0.70. In some embodiments, the antiandrogen is selected from the group consisting of enzalutamide, apalutamide, and darolutamide.

In another aspect, the present disclosure provides a composition comprising any of the herein-described tRNA-pre-miRNA chimeras and an antiandrogen.

In some embodiments, the antiandrogen is selected from the group consisting of enzalutamide, apalutamide, and darolutamide. In some embodiments, the tRNA-pre-miRNA chimera and the antiandrogen act synergistically to inhibit the growth of a Wnt5a-expressing cancer cell. In some embodiments, the cancer cell is a prostate cancer cell. In some embodiments, the co-efficient of drug interaction (CDI) of the tRNA-pre-miRNA chimera and the antiandrogen is less than about 0.95, 0.90, 0.85, 0.80, 0.75, or 0.70. In some embodiments, the tRNA-pre-miRNA chimera is present in an amount effective to reduce or reverse resistance of a cancer cell to antiandrogen. In some embodiments, the cancer cell is a prostate cancer cell.

In another aspect, the present disclosure provides an expression cassette comprising a polynucleotide encoding any of the herein described tRNA-pre-miRNA chimeras, operably linked to a promoter. In another aspect, the present disclosure provides a host cell comprising any of the herein described expression cassettes or tRNA-pre-miRNA chimeras. In some embodiments, the host cell is a bacterial host cell. In some embodiments, the bacterial host cell is E. coli.

In another aspect, the present disclosure provides a method of inhibiting the growth of a Wnt5a-expressing cancer cell, the method comprising contacting the cell with any of the herein-described tRNA-pre-miRNA chimeras or compositions.

In some embodiments of the method, the tRNA-pre-miRNA chimera is processed in the cell, leading to the release of the heterologous Wnt5a-inhibiting RNA sequence in the cell. In some embodiments, the tRNA-pre-miRNA chimera inhibits the expression of Wnt5a in the cell. In some embodiments, the cell is resistant to an antiandrogen, and the method further comprises contacting the cell with antiandrogen. In some embodiments, the tRNA-pre-miRNA chimera and antiandrogen act synergistically to inhibit the growth of the cancer cell. In some embodiments, the co-efficient drug interaction (CDI) of the tRNA-pre-miRNA chimera and the antiandrogen is less than about 0.95, 0.90, 0.85, 0.80, 0.75, or 0.70. In some embodiments, the antiandrogen is selected from the group consisting of enzalutamide, apalutamide, and darolutamide. In some embodiments, the cancer cell is a prostate cancer cell. In some embodiments, the cancer cell is a mammalian cell. In some embodiments, the mammalian cell is a human cell. In some embodiments, the tRNA-pre-miRNA chimera is provided by culturing any of the herein-described host cells, under conditions conducive to the expression of the tRNA-pre-miRNA chimera, and purifying the tRNA-pre-miRNA chimera from the host cell.

In another aspect, the present disclosure provides a method of treating a subject with a Wnt5a-expressing cancer, the method comprising administering to the subject any of the herein-described tRNA-pre-miRNA chimeras or compositions.

In some embodiments, the cancer is resistant to an antiandrogen, and the method further comprises administering the antiandrogen to the subject. In some embodiments, the antiandrogen is selected from the group consisting of enzalutamide, apalutamide, and darolutamide. In some embodiments, the method results in a decrease in the expression of Wnt5a in one or more Wnt5a-expressing cancer cells in the subject. In some embodiments, the method results in a decrease in tumor growth in the subject. In some embodiments, the cancer is prostate cancer. In some embodiments, the method results in a decrease in serum PSA levels in the subject. In some embodiments, the method does not alter the body weight of the subject. In some embodiments, the subject is a human. In some embodiments, the tRNA-pre-miRNA chimera is administered to the subject through intravenous injection. In some embodiments, the tRNA-pre-miRNA chimera is packaged with lipopolyplex prior to administration to the subject.

### BRIEF DESCRIPTION OF THE DRAWINGS

FIG. 1. The workflow for the production of biologic/bioengineered RNAi agent.

FIGS. 2A-2D. FPLC purification of Bioengineered BERA/Wnt5a-siRNA (BERA/Wnt5A-siRNA) molecules. (FIGS. 2A-2B). FPLC traces of BERA/Wnt5a-siRNA2 (FIG. 2A) and BERA/Wnt5a-siRNA1 (FIG. 2B) during the purification. Total RNAs were injected for anion exchange FPLC purification and traces were monitored at 280 nm using a UV/vis detector. (FIG. 2C). Urea-PAGE analysis of unpurified and purified BERA/Wnt5a-siRNA agents. Total RNAs from wild-type and BERA/Wnt5a-siRNA bacteria were used for comparison. (FIG. 2D) HPLC analysis of the purity of isolated BERA/Wnt5a-siRNA agents.

FIGS. 3A-3C. BERA/Wnt5A-siRNA (tRNA-siWint5A) downregulated Wnt5A expression, inhibited CWR22rv1 cell growth and improved enza treatment. FIGS. 3A, 3B: tRNA-siWnt5A-1 and tRNA-siWnt5A-2 inhibited cell growth and improved enza treatment. FIG. 3C: Both Wnt5A siRNA and tRNA-Wnt5A-1, 2 downregulated Wnt5A expression in CWR22rv1 cells.

FIGS. 4A-4E. BERA/Wnt5A-siRNA (tRNA-siWnt5A) inhibited LuCaP35CR tumor growth. FIG. 4A: Tumor volume. Male SCID mice bearing LuCaP35CR PDX tumors were treated with tRNA siWnt5A or tRNA control (LSA) via tail veil injection twice weekly. FIG. 4B: Mice body weight. FIG. 4C: PSA levels in mouse sera at the end of treatment. FIG. 4D: IHC staining of tumor tissues using Wnt5A antibody and Ki67 antibody. FIG. 4E. Quantification of staining in FIG. 4D.

FIGS. 5A-5B. Knockdown of Wnt5a by specific Wnt5a siRNA in C4-2B MDVR (FIG. 5A) and PS1172 CRC (FIG. 5B) cells re-sensitize cells to enzalutamide. Resistant C4-2B MDVR and PS1172 CRC prostate cancer cells were treated with either enzalutamide (Enza) or Wnt5a siRNA (#1) or the combination (#1+Enza) for three days and 5 days, and the cell numbers were determined.

FIGS. 6A-6C. Knockdown of Wnt5a by tRNA-Wnt5a siRNA-1 (tRNA-1) (FIG. 6A) and tRNA-Wnt5a siRNA-2 (tRNA-2) (FIG. 6B) in C4-2B MDVR cells synergizes enzalutamide (ENZA). Resistant C4-2B MDVR prostate cancer cells were treated with either enzalutamide (Enza, 20 uM) or tRNA-1 (10 nM) or tRNA-2 (10 nM) or the combination (combination) for 3 days and 6 days, and the cell numbers were determined. The co-efficient drug interaction (CDI) is shown in FIG. 6C. CDI<1 is considered synergism, especially CDI<0.7 is considered significantly synergistic.

FIGS. 7A-7B. Combination of tRNA Wnt5a with antiandrogens in C4-2B MDVR cells. C4-2B MDVR cells were treated with tRNA Wnt5a and antiandrogens such as apalutamide (Apa), darolutamide (Daro), enzalutamide (enza) individually or combination for 3 days and 6 days and the cell number was determined. The co-efficient drug interaction (CDI) shows below in the table. CDI<1 is considered synergism, especially CDI<0.7 is considered significantly synergistic.

FIGS. 8A-8B. Sequences of the constructs used, i.e., htRNA^{Leu}_pre-miR-34a/Wnt5a-siRNA#1 (FIG. 8A) and htRNA^{Leu}_pre-miR-34a/Wnt5a-siRNA#2 (FIG. 8B). The chimeras use a humanized carrier (using human tRNA) and provides high expression levels and overall yield. Red and green are the siRNA and complementary sequences; underlined is hsa-pre-miR-34a, and the rest is htRNA^{Leu} in which the codon sequence has been replaced with hsa-pre-miR-34a.

FIGS. 9A-9E. Targeting Wnt5A by BERA-Wnt5a siRNA resensitizes LuCaP35CR PDX organoids and tumor growth to enzalutamide treatment. FIG. 9A: Organoids derived from the LuCaP PDX model were established in an ex vivo 3D Matrigel format and treated with bioengineered BERA-Wnt5a siRNA. While organoids remained resistant to enzalutamide treatment in the absence of BERA-Wnt5a siRNA, combinational treatment with BERA-Wnt5a siRNA had robust anti-tumor effects. FIGS. 9B-9C: Tumors from a LuCaP 35CR patient derived xenograft model were resistant to enzalutamide treatment (p>0.05), while a single treatment of BERA-Wnt5a significantly inhibited tumor growth (p<0.05) (FIG. 9C, left). Tumor growth was further suppressed with a combination of BERA-Wnt5a with enzalutamide (p<0.05) (FIG. 9C, right). FIG. 9D: Mouse body weight was unaffected by all of the treatments. FIG. 9E: Immunohistochemical staining of Ki67 also demonstrated that cancer cell proliferation was significantly inhibited by Wnt5a inhibition alone, and that this effect was further enhanced by a combination treatment with enzalutamide.

### DETAILED DESCRIPTION

### 1. Introduction

The present disclosure provides novel compositions and methods involving bioengineered Wnt5A siRNAs or miRNAs (BERA/Wnt5A-siRNA, also referred to herein as, e.g., tRNA-pre-miRNA chimeras) that effectively block Wnt5A expression in cells and inhibit the growth of advanced cancer cells such as prostate cancer cells. The present compositions and therapeutic methods are also effective in overcoming treatment resistance, e.g., resistance to antiandrogens, in subjects, e.g., human subjects.

The present bioengineered Wnt5a siRNAs and miRNAs, which are based upon an optimal tRNA/pre-miRNA carrier, can be produced at high-yield (e.g., >20% of total RNAs) and large-scale (mg of ncRNAs per liter of bacterial culture), allowing the generation of large quantities of highly-purified, biological Wnt5A-siRNA agents (e.g., BERA/Wnt5A-siRNA). The bioengineered tRNAs can be safely used to target gene expression, control human carcinoma cell proliferation and tumor progression. The present tRNA-pre-miRNA chimeras disclosed herein specifically block Wnt5A expression, inhibit cancer cell growth, and can overcome resistance to antiandrogen (e.g., enzalutamide) treatment in vitro and in vivo. BERA/Wnt5A-siRNAs and miRNAs can be used as therapeutics to treat Wnt5A expressing cancers and overcome resistance to therapies, including anti-hormonal therapies.

### 2. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present disclosure. For the purposes of the present disclosure, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “subject,” “individual,” and “patient” as used herein are used interchangeably herein to refer to a vertebrate, preferably a mammal, more preferably a human. Mammals include, but are not limited to, murines, rats, simians, humans, farm animals, sport animals, and pets. Tissues, cells and their progeny of a biological entity obtained in vivo or cultured in vitro are also encompassed.

As used herein, the term “therapeutically effective amount” includes a dosage sufficient to produce a desired result with respect to the indicated disorder, condition, or mental state. The desired result may comprise a subjective or objective improvement in the recipient of the dosage. For example, an effective amount of a tRNA-pre-miRNA chimera includes an amount sufficient to alleviate the signs, symptoms, or causes of cancer, e.g. prostate cancer. As another example, an effective amount of a tRNA-pre-miRNA chimera includes an amount sufficient to prevent the development of a cancer. As another example, an effective amount of a tRNA-pre-miRNA chimera includes an amount sufficient to sensitize antiandrogen-resistant cancer cells to the antiandrogen.

Thus, a therapeutically effective amount can be an amount that slows, reverses, or prevents tumor growth, increases mean time of survival, inhibits tumor progression or metastasis, or re-sensitizes a cancer cell to a cancer drug to which it has become or is resistant (e.g., an antiandrogen drug such as enzalutamide, apalutamide, abiraterone acetate, or bicalutamide). Accordingly, an effective amount of a combination of a tRNA-pre-miRNA chimera and an antiandrogen drug includes an amount sufficient to cause a substantial improvement in a subject having cancer when administered to the subject. In addition, an effective amount of a tRNA-pre-miRNA chimera can include an amount that is effective in enhancing the anti-cancer therapeutic activity of an antiandrogen drug such as enzalutamide, apalutamide, abiraterone acetate, or bicalutamide. The effective amount can vary with the type and stage of the cancer being treated, the type and concentration of one or more compositions administered, and the amounts of other drugs that are also administered.

As used herein, the term “treating” includes, but is not limited to, methods to produce beneficial changes in the health status of a subject, e.g., a cancer patient. The changes can be either subjective or objective and can relate to features such as symptoms or signs of the cancer being treated. For example, if the patient notes decreased pain, then successful treatment of pain has occurred. For example, if a decrease in the amount of swelling has occurred, then a beneficial treatment of inflammation has occurred. Similarly, if the clinician notes objective changes, such as reducing the number of cancer cells, the growth of the cancer cells, the size of cancer tumors, or the resistance of the cancer cells to another cancer drug (e.g., an antiandrogen such as enzalutamide, apalutamide, abiraterone acetate, or bicalutamide), then treatment of cancer has also been beneficial. Preventing the deterioration of a recipient's status is also included by the term. Treating, as used herein, also includes administering a tRNA-pre-miRNA chimera, or a combination of a tRNA-pre-miRNA chimera and an antiandrogen drug (e.g., enzalutamide, apalutamide, abiraterone acetate, bicalutamide, or a combination thereof) to a patient having cancer (e.g., prostate cancer, breast cancer, androgen-independent cancer, metastatic cancer, castrate-resistant cancer, castration recurrent cancer, hormone-resistant cancer, or metastatic castrate-resistant cancer).

As used herein, the term “administering” includes activities associated with providing a patient an amount (e.g., a therapeutically effective amount) of a compound or composition described herein, e.g., a tRNA-pre-miRNA chimera or a combination of a tRNA-pre-miRNA chimera and an antiandrogen drug. Administering includes providing unit dosages of compositions set forth herein to a patient in need thereof. Administering includes providing effect amounts of compounds or compositions described herein for specified period of time, e.g., for about 1, 2, 3, 4, 5, 6, 7, 8, 9, 10, 12, 13, 14, 15, 16, 17, 18, 19, 20, 21, 22, 23, 24, 25, 26, 27, 28, 29, 30, 60, 90, 120, or more days, or in a specified sequence, e.g., administration of a tRNA-pre-miRNA chimera, or administration of a tRNA-pre-miRNA chimera followed by the administration of an antiandrogen drug (e.g., enzalutamide, apalutamide, abiraterone acetate, bicalutamide, or a combination thereof), or vice versa.

The terms “inhibiting,” “reducing,” “decreasing” with respect to tumor or cancer growth or progression refers to inhibiting the growth, spread, metastasis of a tumor or cancer in a subject by a measurable amount using any method known in the art. The growth, progression or spread of a tumor or cancer is inhibited, reduced or decreased if the tumor burden is at least about 10%, 20%, 30%, 50%, 80%, or 100% reduced, e.g., in comparison to the tumor burden prior to administration of a tRNA-pre-miRNA chimera, as described herein, optionally in combination with a chemotherapeutic or anticancer agent. In some embodiments, the growth, progression or spread of a tumor or cancer is inhibited, reduced, or decreased by at least about 1-fold, 2-fold, 3 -fold, 4-fold, or more in comparison to the tumor burden prior to administration of the tRNA-pre-miRNA chimera, optionally in combination with a chemotherapeutic or anticancer agent.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the invention and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like), or for providing the formulation with an edible flavor, etc. One of skill in the art will recognize that other pharmaceutical carriers are useful in the present invention.

As used herein, the term “co-administering” includes sequential or simultaneous administration of two or more structurally different compounds (e.g., a tRNA-pre-miRNA chimera and an antiandrogen drug such as enzalutamide). For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. In some embodiments, the two or more structurally different compounds can be two or more distinct tRNA-pre-miRNA chimeras, i.e., chimeras comprising different inhibitory RNA sequences against Wnt5a (e.g., one comprising the siRNA of SEQ ID NO:3 and one comprising the siRNA of SEQ ID NO:4). In some instances, the co-administered compounds are administered by the same route. In other instances, the co-administered compounds are administered via different routes. For example, one compound can be administered orally, and the other compound can be administered, e.g., sequentially or simultaneously, via intravenous or intraperitoneal injection. The simultaneously or sequentially administered compounds or compositions can be administered such that at least one tRNA-pre-miRNA chimera and one antiandrogen drug are simultaneously present in a subject or in a cell at an effective concentration.

As used herein, the term “cancer” is intended to include any member of a class of diseases characterized by the uncontrolled growth of aberrant cells. The term includes all known cancers and neoplastic conditions, whether characterized as malignant, benign, recurrent, soft tissue, or solid, and cancers of all stages and grades including advanced, recurrent, pre- and post-metastatic cancers. Additionally, the term includes androgen-independent, castrate-resistant, castration recurrent, hormone-resistant, drug-resistant, and metastatic castrate-resistant cancers. Examples of different types of cancer include, but are not limited to, prostate cancer (e.g., prostate adenocarcinoma); breast cancers (e.g., triple-negative breast cancer, ductal carcinoma in situ, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma in situ, Paget's disease, Phyllodes tumors); gynecological cancers (e.g., ovarian, cervical, uterine, vaginal, and vulvar cancers); lung cancers (e.g., non-small cell lung cancer, small cell lung cancer, mesothelioma, carcinoid tumors, lung adenocarcinoma); digestive and gastrointestinal cancers such as gastric cancer (e.g., stomach cancer), colorectal cancer, gastrointestinal stromal tumors (GIST), gastrointestinal carcinoid tumors, colon cancer, rectal cancer, anal cancer, bile duct cancer, small intestine cancer, and esophageal cancer; thyroid cancer; gallbladder cancer; liver cancer; pancreatic cancer; appendix cancer; renal cancer (e.g., renal cell carcinoma); cancer of the central nervous system (e.g., glioblastoma, neuroblastoma); skin cancer (e.g., melanoma); bone and soft tissue sarcomas (e.g., Ewing's sarcoma); lymphomas; choriocarcinomas; urinary cancers (e.g., urothelial bladder cancer); head and neck cancers; and bone marrow and blood cancers (e.g., chronic lymphocytic leukemia, lymphoma). As used herein, a “tumor” comprises one or more cancerous cells.

As used herein, the terms “prostate cancer” and “prostate cancer cell” refer to a cancer cell or cells that reside in prostate tissue or are derived from prostate tissue. In particular embodiments, the prostate cancer cell expresses Wnt5a. The prostate cancer can be benign, malignant, or metastatic. The prostate cancer can be androgen-insensitive, hormone-resistant, or castrate-resistant. The prostate cancer can be “advanced stage prostate cancer” or “advanced prostate cancer.” Advanced stage prostate cancer includes a class of prostate cancers that have progressed beyond early stages of the disease. Typically, advanced stage prostate cancers are associated with a poor prognosis. Types of advanced stage prostate cancers include, but are not limited to, metastatic prostate cancer, drug-resistant prostate cancer such as anti-androgen-resistant prostate cancer (e.g., enzalutamide-resistant prostate cancer, apalutamide-resistant prostate cancer, abiraterone-resistant prostate cancer, bicalutamide-resistant prostate cancer, and the like), taxane-resistant prostate cancer, hormone refractory prostate cancer, castrate-resistant prostate cancer, metastatic castrate-resistant prostate cancer, and combinations thereof. In some instances, the advanced stage prostate cancers do not generally respond, or are resistant, to treatment with one or more of the following conventional prostate cancer therapies: enzalutamide, abiraterone, bicalutamide, or apalutamide. Compounds, compositions, and methods of the present invention are provided for treating prostate cancer, such as advanced stage prostate cancer, including any one or more (e.g., two, three, four, five, six, seven, eight, nine, ten, or more) of the types of advanced stage prostate cancers disclosed herein.

As used herein, the phrase “enhancing the therapeutic effects” includes any of a number of subjective or objective factors indicating a beneficial response or improvement of the condition being treated as discussed herein. For example, enhancing the therapeutic effects of an antiandrogen drug (e.g., enzalutamide, apalutamide, abiraterone acetate, bicalutamide, or a combination thereof) includes re-sensitizing antiandrogen-resistant cancer (e.g., antiandrogen-resistant prostate or breast cancer) to antiandrogen therapy. Also, for example, enhancing the therapeutic effects of an antiandrogen drug (e.g., enzalutamide, apalutamide, abiraterone acetate, bicalutamide, or a combination thereof) includes altering antiandrogen-resistant cancer cells (e.g., antiandrogen-resistant prostate or breast cancer cells) so that the cells are not resistant to the antiandrogen drug (e.g., enzalutamide, apalutamide, abiraterone acetate, bicalutamide, or a combination thereof). Also, enhancing the therapeutic effects of an antiandrogen drug (e.g., enzalutamide, apalutamide, abiraterone acetate, bicalutamide, or a combination thereof) includes additively or synergistically improving or increasing the activity of the antiandrogen drug. In some embodiments, the enhancement includes, or includes at least, about a one-fold, two-fold, three-fold, four-fold, five-fold, ten-fold, twenty-fold, fifty-fold, hundred-fold, or thousand-fold increase in the therapeutic activity of the antiandrogen drug used to treat cancer (e.g., prostate cancer). In some embodiments, the enhancement includes, or includes at least, about a 10%, 20%, 30%, 40%, 50%, 60%, 75%, 80%, 90%, or 100% increase in the therapeutic activity (e.g., efficacy) of the antiandrogen used to treat cancer (e.g., prostate cancer).

As used herein, the terms “reversing cancer cell resistance,” “reducing cancer cell resistance,” or “re-sensitizing cancer cell resistance” to a compound or drug includes altering or modifying a cancer cell that is resistant to a therapy such as antiandrogen therapy (e.g., enzalutamide, abiraterone, bicalutamide, or apalutamide) so that the cell is no longer resistant to antiandrogen therapy, or is less resistant to the antiandrogen therapy. As such, as used herein, the phrase “reversing prostate cancer cell resistance” to an antiandrogen includes altering or modifying a prostate cancer cell that is resistant to an antiandrogen (e.g., enzalutamide, abiraterone, bicalutamide, or apalutamide) therapy so that the cell is no longer resistant to antiandrogen therapy, or is less resistant to the antiandrogen therapy.

As used herein, the phrase “antiandrogen drug” or “antiandrogen” includes antiandrogen compounds that alter the androgen pathway by blocking the androgen receptors, competing for binding sites on the cell's surface, or affecting or mediating androgen production. Antiandrogens are useful for treating several diseases including, but not limited to, cancer (e.g., prostate cancer or breast cancer). Antiandrogen drugs include, but are not limited to, non-steroidal androgen receptor (AR) antagonists and CYP17A1 inhibitors (i.e., androgen synthesis inhibitors that are inhibitors of cytochrome P450 17A1). Non-steroidal AR antagonists include, as non-limiting examples, first-generation drugs (e.g., bicalutamide, flutamide, and nilutamide), second-generation drugs (e.g., apalutamide, darolutamide, and enzalutamide), and others such as cimetidine and topilutamide. Typically, non-steroidal AR antagonists are selective AR antagonists and have little to no antigonadotropic activity. Non-limiting examples of CYP17A1 inhibitors include abiraterone acetate, ketoconazole, and seviteronel.

As used herein interchangeably, a “microRNA,”“miR,” or “miRNA” refers to the unprocessed or processed RNA transcript from a miRNA gene. The unprocessed miRNA gene transcript is also called a “miRNA precursor,” and typically comprises an RNA transcript of about 70-100 nucleotides in length. The miRNA precursor can be processed by digestion with an RNAse (for example, Dicer, Argonaut, or RNAse III) into an active 19-25 nucleotide RNA molecule. This active 19-25 nucleotide RNA molecule is also called the “processed” miRNA gene transcript or “mature” miRNA. The terms “pre-microRNA” or “pre-miR” or pre-miRNA” interchangeably refer to an RNA hairpin comprising within its polynucleotide sequence at least one mature micro RNA sequence (including, in some embodiments, a heterologous mature miRNA or an siRNA sequence) and at least one dicer cleavable site.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g. , the tRNA, pre-miRNA, siRNA, and tRNA-pre-miRNA chimera polynucleotide molecules described herein, e.g., SEQ ID NOs: 1-22 when compared and aligned for maximum correspondence over a comparison window, or designated region as measured using a sequence comparison algorithms (e.g., BLAST, ALIGN, LASTA or any other known alignment algorithm) or by manual alignment and visual inspection. Such sequences are then said to be “substantially identical.” This definition also refers to the compliment of a test sequence. Preferably, the identity exists over a region that is at least about 10, 15, 20, 25, 35, 40, 45, 50, 60, 70, 80, 90, 100, 110, 120 nucleotides in length, or over the full-length of a reference sequence.

“Wnt5a” refers to a ligand of the frizzled family of seven transmembrane receptors. Wnt5a signaling activates the non-canonical (β-catenin-independent) pathway, leading to downstream pathways such as Ca²⁺/calmodulin-dependent protein kinase II, Rho GTPases, G proteins, and JNK kinase. The human Wnt5a sequence can be found, e.g., as UniProt ID P41221, or NCBI Gene ID 7474, the entire disclosures of which are herein incorporated by reference.

As used herein, the term “short interfering nucleic acid” or “siRNA” refers to any nucleic acid molecule capable of down-regulating gene expression in mammalian cells (preferably a human cell). siRNA includes without limitation nucleic acid molecules that are capable of mediating sequence specific RNAi, for example short interfering RNA (siRNA), double-stranded RNA (dsRNA), micro-RNA (miRNA), and short hairpin RNA (shRNA). Likewise, the term “sense region” refers to a nucleotide sequence of an siRNA molecule complementary (partially or fully) to an antisense region of the siRNA molecule. Optionally, the sense strand of a siRNA molecule may also include additional nucleotides not complementary to the antisense region of the siRNA molecule. Conversely, as used herein, the term “antisense region” refers to a nucleotide sequence of a siRNA molecule complementary (partially or fully) to a target nucleic acid sequence. Optionally, the antisense strand of a siRNA molecule may include additional nucleotides not complementary to the sense region of the siRNA molecule. In some embodiments, the sense and antisense strands are also referred to herein as an siRNA sequence and a sequence complementary to an siRNA sequence.

The term “synergy” or “synergistic effect” refers to an effect produced by two or more compounds (e.g., an antiandrogen drug or a tRNA-pre-miRNA chimera as described herein) that is greater than the effect produced by a sum of the effects of the individual compounds (i.e., an effect that is greater than an additive effect). Several methods are available for determining whether a combination of drugs produces a synergistic effect. In some embodiments, the synergism of a combination of compounds (such as a tRNA-pre-miRNA chimera as described herein and an antiandrogen) is determined by calculating the co-efficient of drug interaction (CDI). The CDI can be calculated by determining (E)1,2=E1×E2, where (E)1,2 is the measured combination effect, and E1 and E2 are the drug effects of each agent when applied separately. CDI is calculated by determining CDI=AB/(A×B), where AB is the ratio of the combination groups to the control group, and A or B is the ratio of the single agent group to the control group. A CDI of <1 is considered synergistic, and a CDI of <0.7 indicates that the drug is significantly synergistic.

As an additional, non-limiting example, the Highest Single Agent approach simply reflects that the fact that the resulting effect of a combination of drugs (E_{AB}) is greater than the effects of the individual drugs (E_{A} and E_{B}). A combination index (CI) can be calculated according to the formula:

$$C I = \frac{max ( {E}_{A} , {E}_{B} )}{{E}_{A B}} .$$

As another non-limiting example, according to the Response Additivity Approach, a synergistic drug combination effect occurs when the E_{AB} is greater than the expected additive effects of the individual drugs (E_{A} and E_{B}). Here, the CI is calculated using the formula:

$$C I = \frac{{E}_{A} + {E}_{B}}{{E}_{A B}} .$$

As yet another non-limiting example, the Bliss Independence model is based on the principle that drug effects are the outcomes of probabilistic processes, and makes the assumption that drugs act independently such that they do not interfere with each other (i.e., different sites of action). However, the model also assumes that each drug contributes to the production of a common result. According to this method, the observed combination effect is expressed as a probability (0≤E_{AB}≤1) and is compared to the expected additive effect expressed as

E_{A}+E_{B}(1−E_{A})=E_{A}+E_{B}−E_{A}E_{B},

where 0≤E_{A}≤1 and 0≤E_{B}≤1. The CI for this method is calculated using the formula:

$$C I = \frac{{E}_{A} + {E}_{B} - {E}_{A} {E}_{B}}{{E}_{A B}} .$$

Methods of identifying synergistic effects are further discussed in, e.g., Foucquier J. and Guedj M. Pharmacology Research & Perspectives (2015) (3)3:e00149, which is incorporated herein by reference in its entirety for all purposes.

### 3. tRNA-pre-miRNA Constructs

The tRNA-pre-miRNA chimeras of the present disclosure comprise multiple elements, including a tRNA component, a pre-miRNA component, and a heterologous Wnt5a miRNA or siRNA (present within the pre-miRNA component). The tRNA-pre-miRNA chimeras are constructed such that the pre-miRNA component comprises a heterologous miRNA or siRNA segment, such that upon processing of the chimera in cells, the mature miRNA or siRNA that is released in the cell corresponds to the heterologous miRNA or siRNA. The miRNA or siRNA used in the constructs is directed against Wnt5a, such that the introduction and processing of the chimera into Wnt5a-expressing cells, such as Wnt5a-expressing prostate cancer cells, results in a decrease in the expression of Wnt5a in the cells. The constructs of the present disclosure, e.g., the constructs comprising a tRNA element, a pre-miRNA element, and a Wnt5a inhibitory element inserted within the pre-miRNA element, are interchangeable referred to herein as, e.g., “tRNA-pre-miRNA chimeras,” “tRNA-pre-miRNA molecules,” “tRNA-pre-miRNA constructs,” “tRNA-miRNA chimeras,” “tRNA-miRNA” molecules,” etc.

tRNA-pre-miRNA constructs, and methods of making and using the same, are generally described, e.g., in PCT publications WO2015/183667, WO2019/204733, and WO2019/226603, in U.S. Pat. Nos. 10,619,156 and 10,422,003, and in Chen et al. (2015) Nucl. Acids Res. 43(7):3857-3869; the entire disclosures of each of which are herein incorporated by reference. Any of the tRNA-pre-miRNA constructs, or subsequences thereof, disclosed in any of these publications (including sequences provided in supplementary materials for the publications) can be used in the present disclosure, provided that the pre-miRNA component comprises an miRNA or siRNA sequence against Wnt5a.

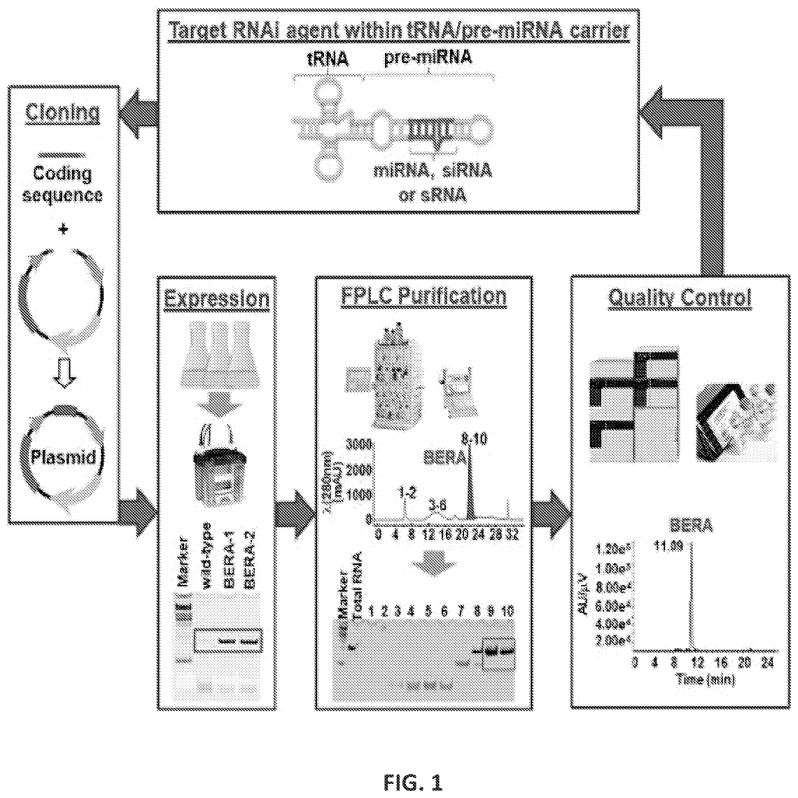

The tRNA-pre-miRNA chimeras of the disclosure comprise two overall structural regions, i.e., a tRNA component and a pre-miRNA component. In some embodiments, the tRNA component is linked to the pre-miRNA by replacing the anticodon of a tRNA with the pre-miRNA molecule, such that the overall RNA molecule (or chimera) comprises, from 5′ to 3′, a first tRNA segment, the pre-miRNA, and a second tRNA segment. In addition, the pre-miRNA sequence comprises an internal heterologous RNA sequence capable of inhibiting Wnt5a, e.g., a Wnt5a miRNA or siRNA sequence. A schematic of the overall structure of the constructs is shown, e.g. in FIG. 1.

tRNA Component

The tRNA component of the chimeras can be any tRNA known in the art, e.g., encoding any amino acid. In some embodiments, the tRNA codes for a leucine. In some embodiments, the tRNA codes for a serine, glycine, glutamate, aspartate, glutamine, arginine, cysteine, lysine, methionine, asparagine, alanine, histidine, isoleucine, phenylalanine, proline, tryptophan, tyrosine, threonine, or valine. In some embodiments, the tRNA is a eukaryotic tRNA, e.g., a mammalian or human tRNA. In some embodiments, the tRNA is a prokaryotic tRNA. tRNAs are well known in the art and a skilled practitioner will be able to select a suitable tRNA for use in the present methods and compositions. The selection of an appropriate tRNA molecule may be, in part, driven by the host cells to be used for expression of the inserted RNA. For example, when seeking to produce high expression levels of a desired inserted RNA molecule, the tRNA selected can be from a tRNA encoding for codon preferred by the species of host cell rather than from a rare codon in the host cell.

The tRNA component will comprise one or more secondary structure elements of tRNAs, e.g., acceptor stem, D arm, variable loop, and T arm. In some embodiments, the tRNA component lacks the stem of the anticodon of the tRNA from which it is derived, with the anticodon region of the tRNA being replaced by the pre-miRNA as described herein. In particular embodiments, accordingly, the tRNA component of the constructs is interrupted and is present in two segments within the construct, e.g., a first tRNA sequence at the 5′ terminus and a second tRNA sequence at the 3′ terminus of the overall RNA molecule. The 5′ and 3′ tRNA sequences or segments can be from the same tRNA (i.e., from the same species and/or coding for the same amino acid) or from different tRNAs (e.g., from different species and/or coding for different amino acids).

In some embodiments, the 5′ (or first) tRNA sequence comprises the sequence shown as SEQ ID NO:9 or SEQ ID NO:10 or a fragment thereof, or the sequence shown as SEQ ID NO:9 or SEQ ID NO:10 with, e.g., 1, 2, 3, 4, 5, 6, 7, 8, 9, 10 or more nucleotide substitutions, or a sequence comprising at least about 80%, 85%, 90%, 91%, 92%, 93%, 94%, 95%, 96%, 97%, 98%, or 99% identity to SEQ ID NO:9 or SEQ ID NO:10, or a fragment thereof. The 5′ tRNA sequence can be any of a variety of lengths, e.g., 20, 25, 30, 35, 40, 41, 42, 43, 44, 45, 46, 47, 48, 49, 50, 51, 52, 53, 54, 55, or more nucleotides.

In some embodiments, the 3′ (or second) tRNA sequence comprises the sequence shown as SEQ ID NO:11 or SEQ ID NO:12 or a fragment thereof, or the sequence shown as SEQ ID NO:11 or SEQ ID NO:12 with, e.g., 1, 2, 3, 4, 5, 6, 7, 8, 9, 10 or more nucleotide substitutions, or a sequence comprising at least about 80%, 85%, 90%, 91%, 92%, 93%, 94%, 95%, 96%, 97%, 98%, or 99% identity to SEQ ID NO:11 or SEQ ID NO:12 or a fragment thereof. The 3′ tRNA sequence can be any of a variety of lengths, e.g., 20, 25, 30, 35, 40, 41, 42, 43, 44, 45, 46, 47, 48, 49, 50, 51, 52, 53, 54, 55, or more nucleotides.

pre-miRNA Sequence

The pre-miRNA component of the chimeras can be derived from any pre-miRNA known in the art, including from natural sources or artificial sources. In some embodiments, the pre-miRNA is derived from pre-miRNA-1291 (see, e.g., miRBase entry MI0006353), human pre-miRNA-34a (MI0000268), human pre-miRNA-125 (MI0000469, MI0000446, MI0000470), human pre-miRNA-124 (MI0000443, MI0000444, MI0000445), human pre-miRNA-27b (MI0000440), human pre-miRNA-22 (MI0000078), pre-let-7c (MI0000064), pre-miR-328 (MI0000804), pre-miR-126 (MI0000471), pre-miR-298 (MI0005523) and pre-miR-200 (MI0000342, MI0000650, MI0000737), and mutants or variants thereof, e.g., having at least about 90%, 91%, 92%, 93%, 94%, 95%, 96%, 97%, 98%, 99% or 100% sequence identity to any of these sequences. In some embodiments, the pre-miRNA is derived from a mammalian pre-miRNA. In some embodiments, the pre-miRNA is derived from a human pre-miRNA.

In particular embodiments, the pre-miRNA is derived from miRNA-34a (see, e.g., NCBI Gene ID No. 407040, or miRBase ID MI0000268, the entire disclosures of which are herein incorporated by reference), e.g., comprises at least about 90%, 91%, 92%, 93%, 94%, 95%, 96%, 97%, 98%, 99% or 100% sequence identity to all or a portion of the full-length miRNA-34a sequence. The overall pre-miRNA region can be of any length, e.g., from about 60 to about 140 nucleotides, or from about 80 to about 120 nucleotides, or about 80, 85, 90, 95, 100, 105, 110, 115, or 120 nucleotides.

In the present tRNA-pre-miRNA chimeras the pre-miRNA sequence comprises an inserted heterologous RNA sequence that, e.g., replaces the endogenous mature miRNA sequence within the pre-miRNA, and that inhibits Wnt5a expression. The inhibitory heterologous RNA sequence targeting Wnt5a is inserted such that processing of the pre-miRNA in a cell releases the mature heterologous RNA sequence, e.g., miRNA or siRNA, in the cell, where it can inhibit Wnt5a expression.

Accordingly, in particular embodiments of the present compositions and methods, the pre-miRNA sequence within the chimeras comprises three regions: a first region extending from the 5′ end of the pre-miRNA to the 5′ end of the heterologous Wnt5a miRNA/siRNA (or complementary sequence, as described below), a second, central region extending from the 3′ end of the heterologous WNt5a miRNA/siRNA (or complementary sequence) to the 5′ end of the sequence complementary to the Wnt5a miRNA/siRNA (or Wnt5a miRNA/siRNA), and a third region extending from the 3′ end of the sequence complementary to the Wnt5a miRNA/siRNA (or the Wnt5a miRNA/siRNA) to the 5′ end of the second (3′) tRNA sequence. The first, second, and third pre-miRNA sequences can be from the same miRNA or from different miRNAs (e.g., from different species and/or derived from different miRNAs).

In particular embodiments, the first pre-miRNA sequence comprises the sequence shown as SEQ ID NO:13 or SEQ ID NO:14 or a fragment thereof, or to a sequence comprising SEQ ID NO:13 or SEQ ID NO:14 with, e.g., 1, 2, 3, 4, 5, 6, 7, 8, 9, 10 or more nucleotide substitutions, or a sequence comprising at least about 80%, 85%, 90%, 91%, 92%, 93%, 94%, 95%, 96%, 97%, 98%, or 99% identity to SEQ ID NO:13 or SEQ ID NO:14 or a fragment thereof. In particular embodiments, the second (central) pre-miRNA sequence comprises the sequence shown as SEQ ID NO:15, SEQ ID NO:16, or SEQ ID NO:17 or a fragment thereof, or to a sequence comprising SEQ ID NO:15, SEQ ID NO:16, or SEQ ID NO:17 with, e.g., 1, 2, 3, 4, 5, 6, 7, 8, 9, 10 or more substitutions, or a sequence comprising at least about 80%, 85%, 90%, 91%, 92%, 93%, 94%, 95%, 96%, 97%, 98%, or 99% identity to SEQ ID NO:15, SEQ ID NO:16, or SEQ ID NO:17 or a fragment thereof. In particular embodiments, the third pre-miRNA sequence comprises the sequence shown as SEQ ID NO:18 or SEQ ID NO:19 or a fragment thereof, or to a sequence comprising SEQ ID NO:18 or SEQ ID NO:19 with, e.g., 1, 2, 3, 4, 5, 6, 7, 8, 9, 10 or more substitutions, or a sequence comprising at least about 80%, 85%, 90%, 91%, 92%, 93%, 94%, 95%, 96%, 97%, 98%, or 99% identity to SEQ ID NO:18 or SEQ ID NO:19 or a fragment thereof.

### WNt5a Inhibiting RNA

The heterologous RNA sequences inserted into the pre-miRNA sequence can be any RNA sequence capable of inhibiting Wnt5a, e.g., a mature microRNA (miRNA), small interfering RNA (siRNA), short hairpin RNA (shRNA) noncoding RNA (ncRNA), Piwi-interacting RNA (piRNA), small nuclear RNA (snRNA), small nucleolar RNA (snoRNA), small activating RNA (saRNA), or catalytic RNA. The pre-miRNA also comprises a sequence complementary to the inhibitory RNA sequence, such that the inhibitory sequence and the complementary sequence can hybridize within the pre-miRNA structure (i.e., a sense and antisense strand). It will be appreciated that, with the inhibitory RNA and the complementary sequence (or sense and antisense strand), as well as with the 5′ and 3′ tRNA sequences and the first and third pre-miRNA sequences, when two sequences are described herein as “complementary,” there is no requirement that the sequences are 100% complementary. The sequences can comprise, e.g., 80%, 85%, 90%, 91%, 92%, 93%, 94%, 95%, 96%, 97%, 98%, or 99% complementary over all or part of the sequence, as long as all or part of the two sequences can hybridize to one another and form, e.g., a double-stranded segment or other secondary structure within the overall RNA molecule. In addition, the miRNA/siRNA and the complementary sequence can be of equivalent size or substantially equivalent size, e.g., their lengths can differ by, e.g., 1, 2, 3, 4 or more nucleotides. The inhibitory sequence and the complementary sequence (or sense and antisense strands) can be present in either order within the pre-miRNA, e.g., in some embodiments the miRNA/siRNA is 5′ of the complementary sequence within the pre-miRNA, and in some embodiments the miRNA/siRNA is 3′ of the complementary sequence. The inhibitory RNA sequence can be of any length, e.g., from about 15 to about 45 nucleotides, or from about 18 to about 30 nucleotides, or from about 20 to 25 nucleotides, or 18, 19, 20, 21, 22, 23, 24, 25, 26, 27, 28, 29 or more nucleotides.

In particular embodiments, the Wnt5a inhibiting component is an siRNA against Wnt5a. Designing siRNA sequences against a target gene, i.e., a Wnt5a mRNA, is well known in the art and any suitable sequence can be inserted into the tRNA-pre-miRNA constructs of the invention. In particular embodiments, the Wnt5a siRNA comprises the sequence shown as SEQ ID NO:3, SEQ ID NO:4, SEQ ID NO:5, or SEQ ID NO:6 or a fragment thereof, or a sequence comprising SEQ ID NO:3, SEQ ID NO:4, SEQ ID NO:5, or SEQ ID NO:6 with, e.g., 1, 2, 3, 4, or more mismatches, or a sequence comprising at least about 80%, 85%, 90%, 91%, 92%, 93%, 94%, 95%, 96%, 97%, 98%, or 99% identity to SEQ ID NO:3, SEQ ID NO:4, SEQ ID NO:5, or SEQ ID NO:6 or a fragment thereof, so long that the siRNA is capable of silencing Wnt5a expression in cells. In particular embodiments, the complementary Wnt5a siRNA comprises the sequence shown as SEQ ID NO:7 or SEQ ID NO:8 or a fragment thereof, or a sequence comprising SEQ ID NO:7 or SEQ ID NO:8 with, e.g., 1, 2, 3, 4, 5, or more mismatches, or a sequence comprising at least about 80%, 85%, 90%, 91%, 92%, 93%, 94%, 95%, 96%, 97%, 98%, or 99% identity to SEQ ID NO:7 or SEQ ID NO:8 or a fragment thereof.

In particular embodiments, the tRNA-pre-miRNA chimera comprises the sequence shown as SEQ ID NO:20 or SEQ ID NO:21 or a fragment thereof, or a sequence comprising at least about 80%, 85%, 90%, 91%, 92%, 93%, 94%, 95%, 96%, 97%, 98%, or 99% identity to SEQ ID NO:20 or SEQ ID NO:21 or a fragment thereof. In some embodiments, the tRNA-pre-miRNA chimera comprises the sequence shown as SEQ ID NO:22 or a fragment thereof, or a sequence comprising at least about 80%, 85%, 90%, 91%, 92%, 93%, 94%, 95%, 96%, 97%, 98%, or 99% identity to SEQ ID NO:22 or a fragment thereof, wherein (N1) corresponds to the Wnt5a siRNA or miRNA sequence, of length n, and (N2) corresponds to the complementary Wnt5a siRNA or miRNA sequence, also of length n.

### Variants and Derivatives

In some embodiments, the tRNA-pre-miRNA constructs of the present disclosure comprise one or more chemical modifications or modified ribonucleotide bases. For example, the constructs can comprise, inter alia, internucleotide linkages, internucleoside linkages, dideoxyribonucleotides, 2′-sugar modification, 2′-amino groups, 2′-fluoro groups, 2′-methoxy groups, 2′-alkoxy groups, 2′-alkyl groups, 2′-deoxyribonucleotides, 2′-O-methyl ribonucleotides, 2′-deoxy-2′-fluoro ribonucleotides, universal base nucleotides, acyclic nucleotides, 5-C-methyl nucleotides, biotin groups, terminal glyceryl incorporation, inverted deoxy abasic residue incorporation, sterically hindered molecules, 3′-deoxyadenosine (cordycepin), 3′-azido-3′-deoxythymidine (AZT), 2′,3′-dideoxyinosine (ddl), 2′,3′-dideoxy-3′-thiacytidine (3TC), 2′,3′-didehydro-2′,3′-dideoxythymidi-ne (d4T), monophosphate nucleotide modification (MNM) of 3′-azido-3′-deoxythymidine (AZT), MNM-2′,3′-dideoxy-3′-thiacytidine (3TC), MNM-2′,3′-didehydro-2′,3′-dide-oxythymidine (d4T), capping moieties, L-nucleotides locked nucleic acid (LNA) nucleotides, 2′-methoxyethoxy (MOE) nucleotides, 2′-methyl-thio-ethyl, 2′-deoxy-2′-fluoro nucleotides, 2′-deoxy-2′-chloro nucleotides, 2′-azido nucleotides, 2′-O-methyl, cholesterol groups, 2′-O-methyl groups, phosphorothioate groups, 2′-fluoro groups, 2′-O-methyoxyethyl groups, boranophosphate groups, 4′-thioribose groups, bile acid, lipids, and bridges connecting the 2′-oxygen and 4′-carbon. Ribonucleotide analogs are described, e.g., in Sprinzl et al. (1998) “Compilation of tRNA sequences and sequences of tRNA genes”. Nucleic Acids Res., 26, 148-153 and on the basis of “RNA modification database” data (medstat.med.utah.edu/RNAmods/), and can include, e.g., 1-methyl-A, inosine, 2′-O-methyl-A, 5-methyl-C, 2′-O-methyl-C, 7-methyl-G, 2′-O-methyl-G pseudouridine, ribothymidine, 2′-O-methyl-ribothymidine, dihydrouridine, 4-thiouridine, 3 -(3 -amino-3 -carboxypropyl)-uridine. ribothymidine, 2′-O-methyl-ribothymidine, dihydrouridine, 4-thiouridine, and 3-(3-amino-3-carb oxypropyl)-uridine.

### Assessing Inhibition of Wnt5a Expression

Any of a number of methods can be used to assess the level of Wnt5a in cells or tissues, e.g., for assessing the efficacy of a tRNA-mi-preRNA chimera as described herein in inhibiting Wnt5a expression. For example, the level of Wnt5a can be assessed by examining the transcription of a gene encoding Wnt5a (e.g., the WNTSA gene; see, e.g., NCBI Gene ID No. 7474), by examining the levels of Wnt5a protein, by measuring Wnt5a signaling activity, or indirectly by measuring, e.g., the growth of Wnt5a-expressing prostate cancer cells.

In some embodiments, the methods involve the detection of Wnt5a-encoding polynucleotide (e.g., mRNA) expression, which can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the methods involve the detection of Wnt5a protein expression, e.g., using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (META); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

### 4. Producing tRNA-pre-miRNA Chimeras

The tRNA-pre-miRNA chimeras of the present disclosure can be prepared in any of a number of ways. In some embodiments, the chimeras are synthesized, e.g., in the laboratory using an oligo synthesizer, e.g., as sold by Applied Biosystems, Biolytic Lab Performance, Sierra Biosystems, or others. Alternatively, RNA molecules with any desired sequence and/or modification can be readily ordered from any of a large number of suppliers, e.g., ThermoFisher, Biolytic, IDT, Sigma-Aldritch, GeneScript, etc.

In particular embodiments, the tRNA-pre-miRNA chimeras are produced recombinantly, i.e., by introducing an expression vector encoding the chimeras into cells wherein the chimera can be expressed and subsequently purified. The cells used for recombinant expression can be prokaryotic or eukaryotic. In some embodiments, the cells used for the expression of the chimeras are from the same species as the tRNA or pre-miRNA component of the chimeric molecule. In particular embodiments, the cells used to express the tRNA-pre-miRNA chimeras do not comprise an endonuclease capable of cleaving out the heterologous miRNA or siRNA from the pre-miRNA sequence, e.g., Dicer. In some embodiments, the tRNA-pre-miRNA chimeras are produced in eukaryotic cells, such as mammalian cells, human cells, plant cells, yeast cells, or others. In particular embodiments, the tRNA chimeras are produced in bacteria, e.g., E. coli. The chimeras can be produced at high yield (e.g., more than 23% of total RNAs) and at a large scale (e.g., mg of chimera RNAs per liter of bacterial culture).

To obtain high level expression of a nucleic acid encoding a tRNA-pre-miRNA chimera of the present disclosure, a polynucleotide encoding the chimera can be subcloned into an expression vector that contains a strong promoter (typically heterologous) to direct transcription and a transcription terminator. Suitable bacterial promoters are well known in the art and described, e.g., in Sambrook and Russell, supra, and Ausubel et al., supra. Bacterial expression systems for expressing a recombinant polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function. The promoter can be a constitutive or an inducible promoter.

In addition to a promoter sequence, the expression cassette should also contain a transcription termination region to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, pET30(a)+, and fusion expression systems such as GST and LacZ.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the peptide under the direction of the polyhedrin promoter or other strong baculovirus promoters.

The elements that are typically included in expression vectors also include a replicon that functions in E. coli, a gene encoding a protein that provides antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

### Transfection and Purification

Standard transfection methods are used to produce bacterial, mammalian, yeast, insect, or plant cell lines that express large quantities of a tRNA-pre-miRNA chimera as described herein, which is then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells is performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

In some embodiments, the tRNA-pre-miRNA chimeras are purified as part of the total RNA from host cells. Methods of isolating or purifying total RNA from a host cell are established in the art. Methods that can be used include, e.g., separation by gel electrophoresis, affinity chromatography, chromatography, FPLC and/or HPLC. In some embodiments, the substantially isolated and/or purified tRNA-pre-miRNA chimeras are then transfected or delivered into a eukaryotic cell, which will then process the tRNA-pre-miRNA chimeras to release the inserted heterologous siRNA or miRNA. In some embodiments, the tRNA-pre-miRNA chimeras are contacted with or exposed to an endoribonuclease (e.g., Dicer) in vitro, under conditions sufficient to allow cleave or release of the inserted heterologous RNA. If desired, the in vitro cleavage or release of the inserted heterologous RNA can be facilitated, e.g., by adding an RNase or DNAzyme site to the tRNA-pre-miRNA molecule. In particular embodiments, the tRNA-pre-miRNA chimeras are purified and then introduced into cells, e.g., prostate cancer cells, or administered to a subject as described in more detail elsewhere herein.

### 5. Administration and Formulation

### Subjects

The herein-described tRNA-pre-miRNA chimeras can be administered to a subject in need thereof (e.g., a subject diagnosed as having cancer, e.g., prostate cancer) for the ultimate delivery of a heterologous Wnt5a-inhibiting RNA of interest to the interior of a target cell. Generally, the subject is a mammal and therefore comprises eukaryotic cells which express endoribonucleases (e.g., Dicer). Once the target eukaryotic cells of the subject have been transfected or transformed with the tRNA-pre-miRNA chimeras, the endoribonucleases (e.g., Dicer) within the target cell cleave out or release the inserted Wnt5a-inhibiting RNA, which can then inhibit Wnt5a expression in the cell. In particular embodiments, the subject has prostate cancer in which some or all of the cancer cells express Wnt5a. In particular embodiments, the prostate cancer is resistant to one or more antiandrogens, e.g., enzalutamide.

The subject can be any subject, e.g. a human or other mammal, that has a Wnt5a-expressing cancer, e.g., prostate cancer, or that is at risk of developing a Wnt5a-expressing cancer. In some embodiments, the subject is a human. In some embodiments, the subject is an adult. In some embodiments, the subject is an adolescent. In some embodiments, the subject is a child. In some embodiments, the subject is female (e.g., an adult or adolescent female). In some embodiments, the subject is male (e.g., an adult or adolescent male).

### Formulations

The present disclosure provides pharmaceutical compositions comprising a tRNA-pre-miRNA chimera and a pharmaceutically acceptable carrier. Suitable formulations include liposomal formulations and combinations with other agents or vehicles/excipients such as cyclodextrins which may enhance delivery of the RNA. In some embodiments, suitable carriers include lipid-based carriers such as a stabilized nucleic acid-lipid particle (e.g., SNALP or SPLP), cationic lipid or liposome nucleic acid complexes (i.e., lipoplexes), a liposome, a micelle, a virosome, or a mixture thereof. In other embodiments, the carrier system is a polymer-based carrier system such as a cationic polymer-nucleic acid complex (i.e., polyplex). In alternative embodiments, the carrier system is a cyclodextrin-based carrier system such as a cyclodextrin polymer-nucleic acid complex. In further embodiments, the carrier system is a protein-based carrier system such as a cationic peptide-nucleic acid complex.

Colloidal dispersion systems, such as macromolecule complexes, nanocapsules, microspheres, beads, and lipid-based systems including oil-in-water emulsions, micelles, mixed micelles, and liposomes, may be used as delivery vehicles for the tRNA-pre-miRNA chimeras described herein. Commercially available fat emulsions that are suitable for delivering the nucleic acids to tissues, such as cardiac muscle tissue and smooth muscle tissue, include Intralipid, Liposyn, Liposyn II, Liposyn III, Nutrilipid, and other similar lipid emulsions. Exemplary formulations are also disclosed in U.S. Pat. No. 5,981,505; 6,217,900; 6,383,512; 5,783,565; 7,202,227; 6,379,965; 6,127,170; 5,837,533; 6,747,014; and WO03/093449, Ohosh and Bachhawat, 1991, which are herein incorporated by reference in their entireties. In some embodiments, the tRNA-pre-miRNA chimeras are complexed with a polyethylenimine (PEI), e.g., liposomal-branched polyethylenimine (PEI) polyplex (LPP) or in vivo-jetPEI (IPEI). In some embodiments, the tRNA-pre-miRNA construct is complexed with a branched polyethylenimine, e.g., with an average molecular weight of about 10,000 Da. The complex can then be encapsulated in a lipid bilayer, e.g., comprising a mixture of 1,2-di-0-octadecenyl-3-trimethylammonium propane (DOTMA), cholesterol and 1,2-Dimyristoyl-sn-glycerol, methoxypolyethylene glycol (DMG-PEG2000).

In some embodiments, liposomes are complexed with a hemagglutinating virus (HVJ), to facilitate fusion with the cell membrane and promote cell entry of liposome-encapsulated DNA (Kaneda et al., 1989). In other embodiments, the liposomes are complexed or employed in conjunction with nuclear non histone chromosomal proteins (HMG-I) (Kato et al., 1991). In some embodiments, the liposomes are complexed or employed in conjunction with both HVJ and HMG-I.

In particular embodiments, the tRNA-pre-miRNA constructs are packaged and delivered using lipopolyplex (LPP) (see, e.g., Bofinger et al., (2018) doi.org/10.1002/psc.3131), Ewe & Algner (2016) Meth. Mol. Biol. (Doi 10.1007/978-1-4939-3718-9_12), the entire disclosures of which are herein incorporated by reference.

Therapeutic formulations may be in the form of liquid solutions or suspensions. For enteral administration, the compound may be administered in a tablet, capsule or dissolved in liquid form. The table or capsule may be enteric coated, or in a formulation for sustained release. For intranasal formulations, in the form of powders, nasal drops, or aerosols. Suitable formulations include those that have desirable pharmaceutical properties, such as targeted delivery to cancer cells, improved serum half-life/stability of a tRNA-pre-miRNA chimera, improved intracellular penetration and cytoplasmic delivery, improved persistence of in-vivo activity, reduction in dose required for efficacy, reduction in required dosing frequency, etc. In some embodiments, a gene therapy approach for the transduction of polynucleotides encoding a tRNA-pre-miRNA chimera to target cells (e.g., prostate cancer cells) using for example lentiviral-based vectors, may be used.

Methods well known in the art for making formulations are found in, for example, Remington: the Science & Practice of Pharmacy, Loyd, et al., eds., 22nd ed., Pharmaceutical Press, (2012). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9- lauryl ether, glycocholate and deoxycholate, or may be oily solutions for administration in the form of nasal drops, or as a gel. For therapeutic or prophylactic compositions, the tRNA-pre-miRNA chimeras (or a composition comprising a tRNA-pre-miRNA chimera and an antiandrogen) are administered to an individual in an amount sufficient to stop or slow a cancer, to promote differentiation, to inhibit or decrease self-renewal, to sensitize a cancer cell to an antiandrogen, or to inhibit or decrease engraftment or metastasis of cancer cells.

Pharmaceutical forms suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

### Antiandrogens

In some embodiments, the tRNA-pre-miRNA chimeras are administered as a composition also comprising an antiandrogen, e.g., to a subject with Wnt5a-expressing, antiandrogen-resistant prostate cancer. Accordingly, the present disclosure provides compositions comprising a tRNA-pre-miRNA chimera as described herein and an antiandrogen. Also provided are methods of treating prostate cancer in a subject, comprising administering to the subject a tRNA-pre-miRNA chimera as described herein and an antiandrogen.

The antiandrogen in the composition and/or used in the present methods can be any antiandrogen, including steroidal and non-steroidal antiandrogens, e.g., enzalutamide, bicalutamide, abiraterone, flutamide, nilutamide, apalutamide, darolutamide, proxalutamide, cimetidine, topilutamide, 17α-Hydroxyprogesterone derivatives such as dhlormadinone acetate, cyproterone acetate, megestrol acetate, and osaterone acetate, 19-Norprogesterone derivatives such as nomegestrol acetate, 19-Nortestosterone derivatives such as dienogest and oxendolone, 17α-Spirolactone derivatives such as drospirenone and spironolactone, medrogestone, and others.

In some embodiments, the tRNA-pre-miRNA chimera and antiandrogen used in the composition or method act synergistically to inhibit the growth of cancer cells, e.g., Wnt5a-expressing prostate cancer cells. In some embodiments, the tRNA-pre-miRNA chimera and antiandrogen have a coefficient of drug interaction (CDI) of less than about 0.95, 0.90, 0.85, 0.75, 0.70, or lower. In some embodiments, the tRNA-pre-miRNA chimera used in the composition or method is present in an amount effective to reduce or reverse resistance of the cancer cell to the antiandrogen. In some embodiments, the tRNA-pre-miRNA chimera used in the composition or method is present in an amount effective to resensitize the cancer cell to the antiandrogen.

### Administration

The formulations as described herein may be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous, intrahepatic, intratumoral and intraperitoneal administration. Preferably, sterile aqueous media are employed as is known to those of skill in the art. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Conventional pharmaceutical practice may be employed to provide suitable formulations or compositions to administer the tRNA-pre-miRNA chimeras to subjects suffering from, at risk of, or presymptomatic for cancer, e.g., prostate cancer. Suitable pharmaceutical compositions may be formulated by means known in the art and their mode of administration and dose determined by the skilled practitioner. Any appropriate route of administration may be employed, for example, parenteral, intravenous, subcutaneous, intramuscular, intraventricular, intraurethral, intraperitoneal, intrahepatic, intratumoral, intranasal, aerosol, oral administration, or any mode suitable for the selected treatment.

The tRNA-pre-miRNA chimeras may be provided alone or in combination with other compounds (for example, an antiandrogen or a chemotherapeutic agent), in the presence of a liposome, an adjuvant, or any pharmaceutically acceptable carrier, in a form suitable for administration to mammals, for example, humans, cattle, sheep, etc. If desired, treatment with the tRNA-pre-miRNA chimeras may be combined with other therapies for cancer, e.g., targeted chemotherapies using cancer-specific peptides described, e.g., in intl. Publ. No. 2011/038142.

The hybrid tRNA-pre-miRNA chimeras may be administered chronically or intermittently. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature. In some embodiments, the tRNA-pre-miRNA chimera is administered to a subject in need thereof, e.g., a subject diagnosed with or suspected of having a cancer, e.g., a Wnt5a-expressing prostate cancer.

In some embodiments, a tRNA-pre-miRNA chimera is delivered to cancer cells, by a variety of methods known to those skilled in the art. Such methods include but are not limited to liposomal encapsulation/delivery, vector-based gene transfer, fusion to peptide or immunoglobulin sequences (peptides described, e.g. , in Intl. Publ. No. 2011/038142) for enhanced cell targeting and other techniques.

An “effective amount” of a tRNA-pre-miRNA chimera includes a therapeutically effective amount or a prophylactically effective amount. A “therapeutically effective amount” refers to an amount effective, at dosages and for periods of time necessary, to achieve the desired therapeutic result, such as treatment of a cancer or promotion of differentiation, or inhibition or decrease of self-renewal or inhibition or decrease of engraftment or metastasis of a cancer cell. The increase or decrease may be between 10% and 90%, e.g., 10%, 20%, 30%, 40%, 50%, 60%, 70%, 80%, 90%, or may be over 100%, such as 200%, 300%, 500% or more, when compared with a control or reference subject, sample or compound.

A therapeutically effective amount of a tRNA-pre-miRNA chimera may vary according to factors such as the disease state, age, sex, and weight of the individual subject, and the ability of the tRNA-pre-miRNA chimera to elicit a desired response in the individual. Dosage regimens may be adjusted to provide the optimum therapeutic response. A therapeutically effective amount is also one in which any toxic or detrimental effects of the tRNA-pre-miRNA chimera are outweighed by the therapeutically beneficial effects. A “prophylactically effective amount” refers to an amount effective, at dosages and for periods of time necessary, to achieve the desired prophylactic result, such as prevention or protection against a cancer or promotion of differentiation, inhibition or decrease of self-renewal or inhibition or decrease of engraftment or metastasis of cancer cells. Typically, a prophylactic dose is used in subjects prior to or at an earlier stage of disease, so that a prophylactically effective amount may be less than a therapeutically effective amount. In alternative embodiments, dosages may be adjusted depending on whether the subject is in remission from cancer or not. A preferred range for therapeutically or prophylactically effective amounts of a tRNA-pre-miRNA chimera may be any integer from 0.1 nM-0.1 M, 0.1 nM-0.05 M, 0.05 hM-15 mM or 0.01 hM-10 pM. In some embodiments, a therapeutically or prophylactically effective amount that is administered to a subject may range from about 5 to about 3000 micrograms/kg if body weight of the subject, or any number therebetween.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

### 6. Kits

The present disclosure also provides kits comprising tRNA-pre-miRNA chimeras as described herein. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kit comprises one or more reagents for the treatment of a subject with Wnt5a-expressing prostate cancer. In some embodiments, the kit further comprises an antiandrogen. In some embodiments, the kit further comprises one or more plasmid, bacterial or viral vectors for expression of the polynucleotide encoding a tRNA-pre-miRNA chimera. In some embodiments, the kit further comprises one or more additional therapeutic agents, e.g., a chemotherapeutic agent used to treat cancer, e.g., prostate cancer.

In some embodiments, the kits can further comprise instructional materials containing directions (i.e., protocols) for the practice of the methods of this invention (e.g., instructions for using the kit for inhibiting or slowing the growth of cancer cells, for treating cancer, for inhibiting the expression of Wnt5a in a cell, etc.) While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### 7. Examples

### Example 1. Preparation of Constructs and In Vitro and In Vivo Assays

Motivated by the idea of producing biological RNAs to perform RNA actions, we used

a novel RNA bioengineering technology to achieve high-yield (e.g., 10-20% of total RNAs) and large-scale (mg of ncRNAs per liter of bacterial culture) production of biological miRNA/siRNA agents, which is based upon an optimal tRNA/pre-miRNA noncoding RNA scaffold (OnRS) (FIG. 1). The bioengineered tRNAs have shown their activity in the control of human carcinoma cell proliferation, target gene expression, xenograft tumor progression, and safety profiles. We generated large quantities of highly-purified, biological Wnt5A-siRNA agents based on this novel RNA bioengineering technology. We identified effective Wnt5A-siRNA sequences (e.g., 5′-ACAAACUGGUCCACGAUCUCCGUGC-3′ and 5′-CUAGGAAGAACUUGGAAGACAUUGC-3′, etc.). We constructed corresponding Wnt5A-siRNA expression plasmids through molecular cloning. We detected and verified the expression of recombinant ncRNAs using a direct, reliable, and practical urea-PAGE assay, following the isolation of total RNAs and spectrometric analysis. In addition, we quantitatively analyzed the levels of target ncRNAs accumulated within E. coli using qPCR assays. Wild-type cells and cells transformed with tRNA expression plasmids were used as controls. We purified recombinant Wnt5A-siRNA agents (tRNA-siWnt5A-1, -2) to high homogeneity (>95%) using an established anion exchange FPLC method.

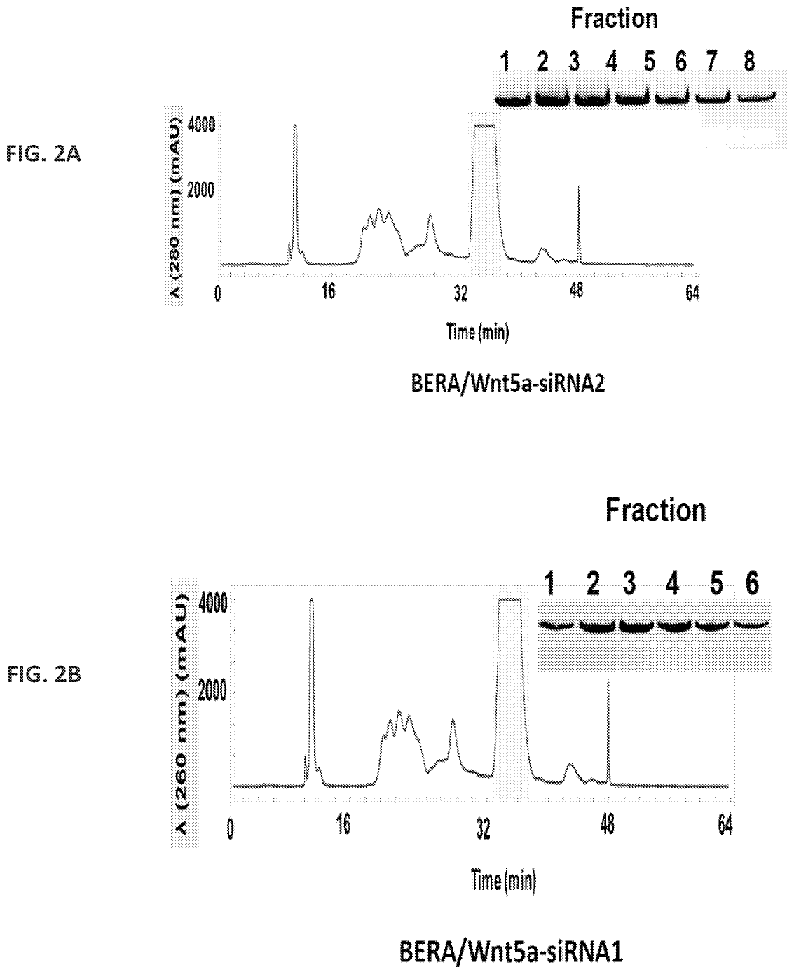

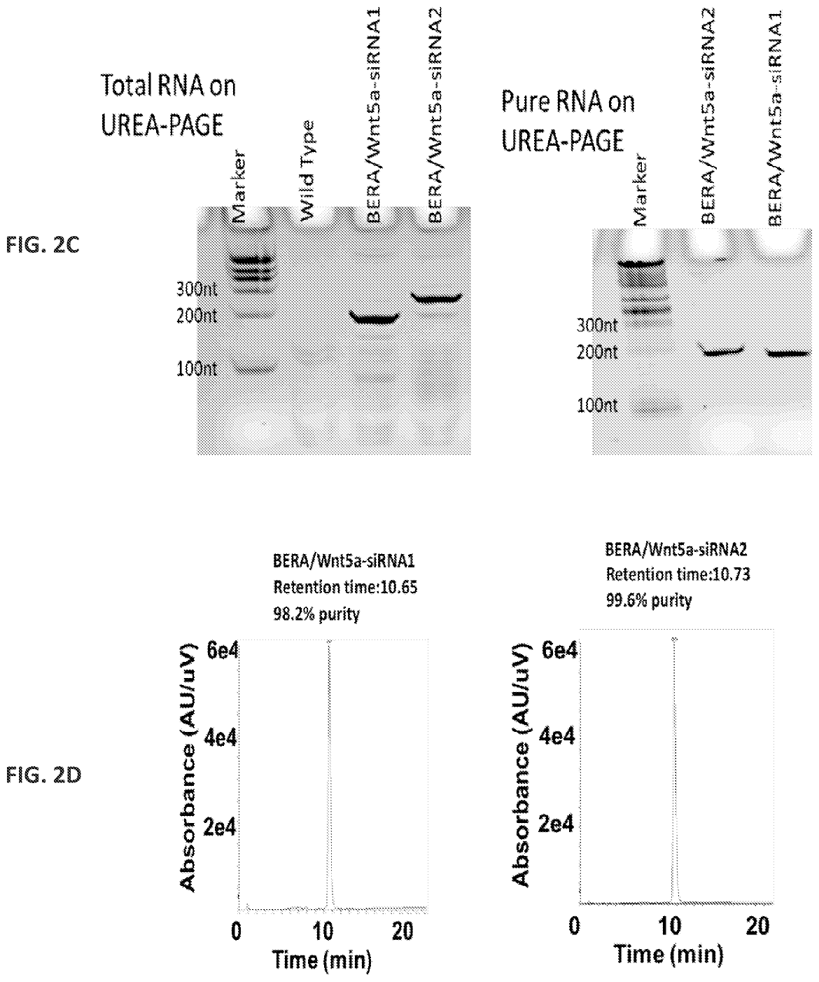

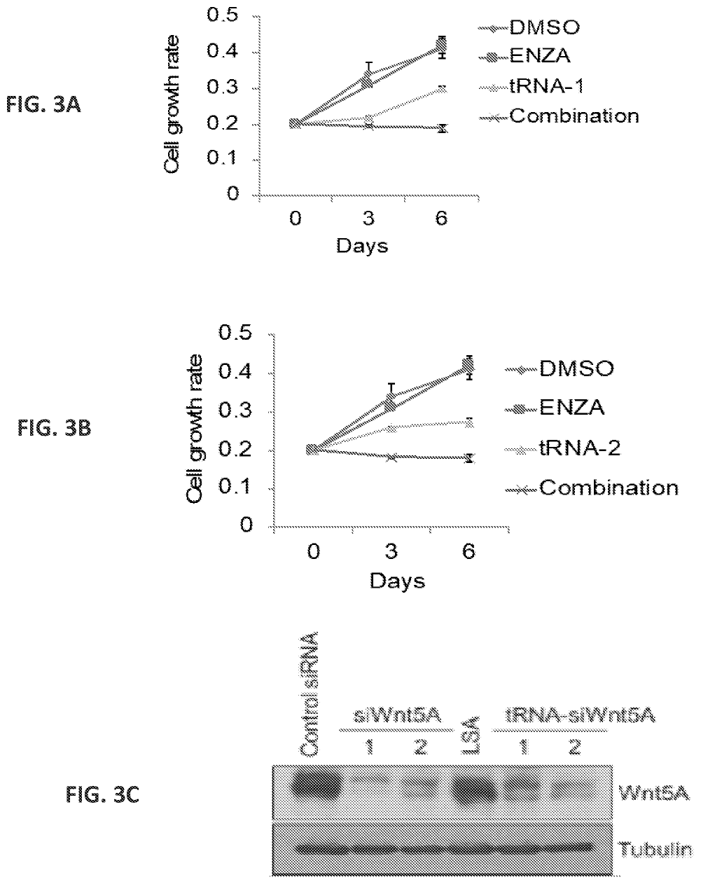

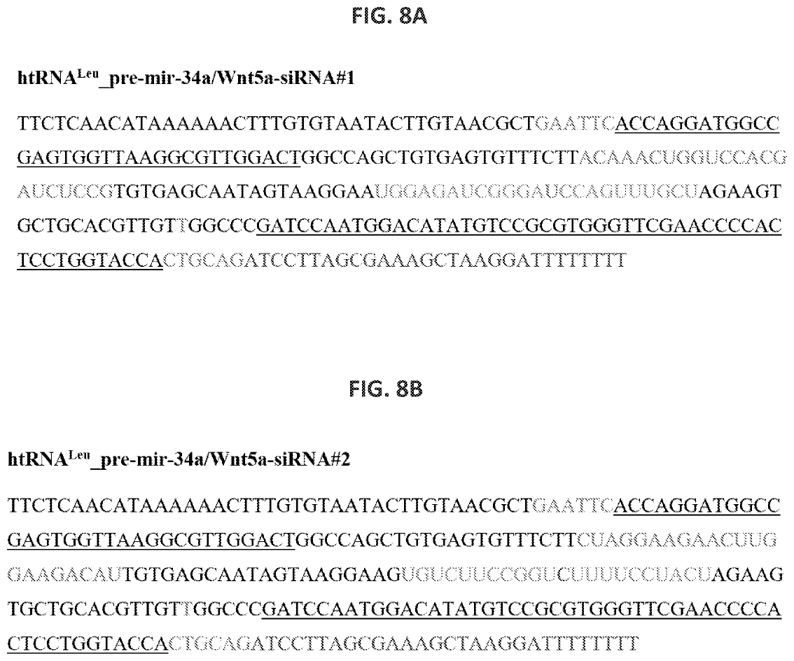

In addition to spectrometry and gel electrophoresis analyses of target Wnt5A-siRNAs during the purification described above, we conducted HPLC analysis to validate the purity of isolated siRNAs and LC-MS studies to verify RNA sequence and identify possible posttranscriptional modifications. We purified bioengineered siWnt5A agents BERA/Wnt5A-siRNA (tRNA-siWnt5A-1 (see, e.g., FIG. 8A) and tRNA-siWnt5A-2 (see, e.g., FIG. 8B) based on these protocols, as shown in FIGS. 2A-2D. Functional studies showed that both BERA/Wnt5A-siRNA (tRNA-siWnt5A-1 and tRNA-siWnt5A-2) downregulated Wnt5A expression and inhibited enzalutamide resistant CWR22rv1 cell growth and improved enzalutamide treatment (FIGS. 3A-3C).

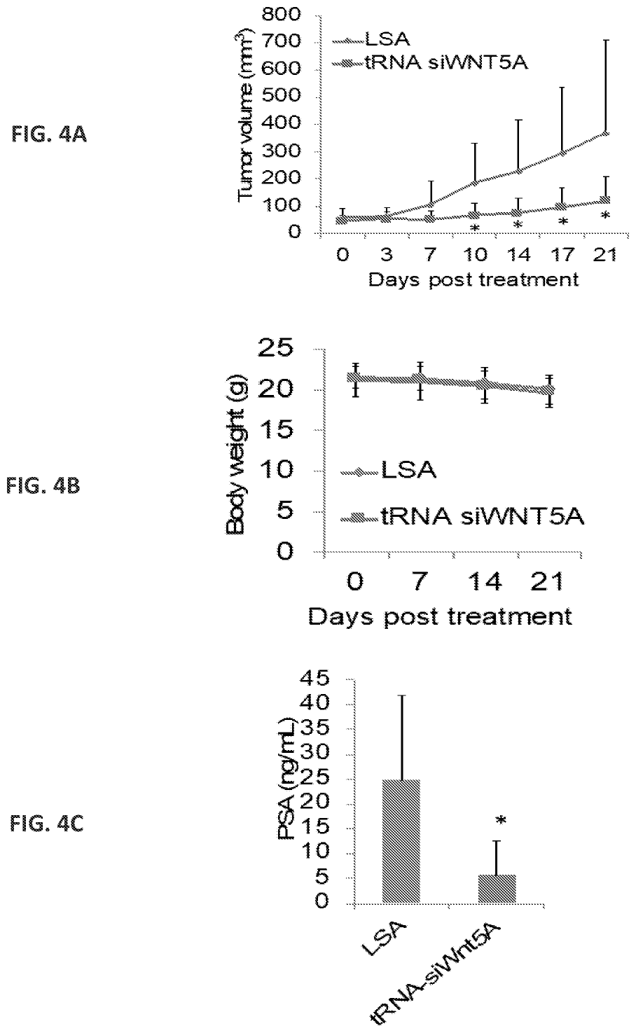

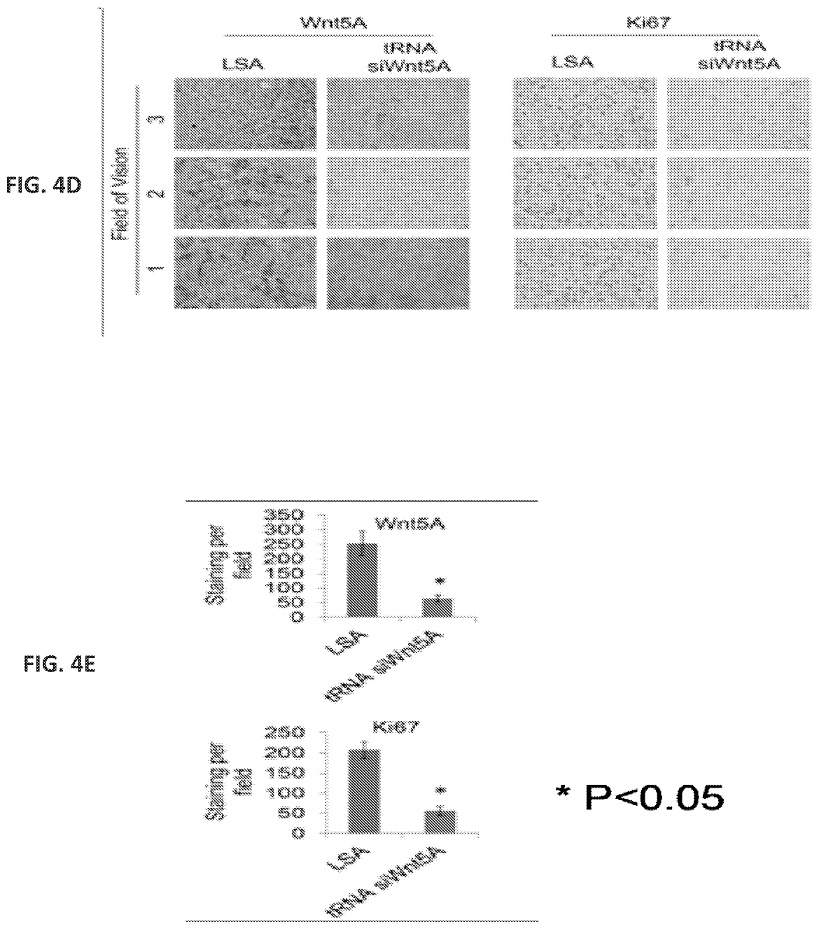

To further characterize the effects of BERA/Wnt5A-siRNA (tRNA-siWnt5A) on tumor growth in vivo, the LuCaP35 CR model was used. Briefly, 3-4 weeks C.B17/lcrHsd-Prkdc-SCID mice (ENVIGO) were surgically castrated. Two weeks later, ˜20- to 30-mm3 pieces of LuCaP tumor were implanted into the pre-castrated SCID mice. When tumors reached 50-100 mm³, mice were randomized into two groups and treated as follows through intravenous (i.v.) injection: (1) LSA (30 μg/mouse), (2) BERA/Wnt5A-siRNA (tRNA-siWnt5A) (30 μg/mouse), the LSA and BERA/Wnt5A-siRNA (tRNA-siWnt5A) were packaged with lipopolyplex (LPP) immediately before use. Tumors were measured using calipers twice a week and tumor volumes were calculated using length×width×width×0.52. Tumor tissues were harvested and weighed after 3 weeks of treatment. Serum was collected for PSA determination. As shown in FIG. 4A, BERA/Wnt5A-siRNA (tRNA-siWnt5A) significantly suppressed LuCaP 35CR growth and tumor weight. Treatments did not alter mouse body weights (FIG. 4B). BERA/Wnt5A-siRNA (tRNA-siWnt5A) treatment also significantly suppressed serum PSA level (FIG. 4C). Immunohistochemical staining of Wnt5A showed BERA/Wnt5A-siRNA (tRNA-siWnt5A) decreased Wnt5A expression in tumors (FIGS. 4D-4E). Additionally, Ki67 staining showed cell proliferation was significantly inhibited by BERA/Wnt5A-siRNA (tRNA-siWnt5A) treatment (FIGS. 4D-4E). Taken together, these results demonstrate that inhibition of Wnt5A by bioengineered tRNA-siWnt5A (BERA/Wnt5A-siRNA) reduces enzalutamide-resistant tumor growth with minimal toxicity.

### Example 2. tRNA-pre-miRNA Chimeras Act Synergistically With Antiandrogens to Inhibit Cancer Cell Growth

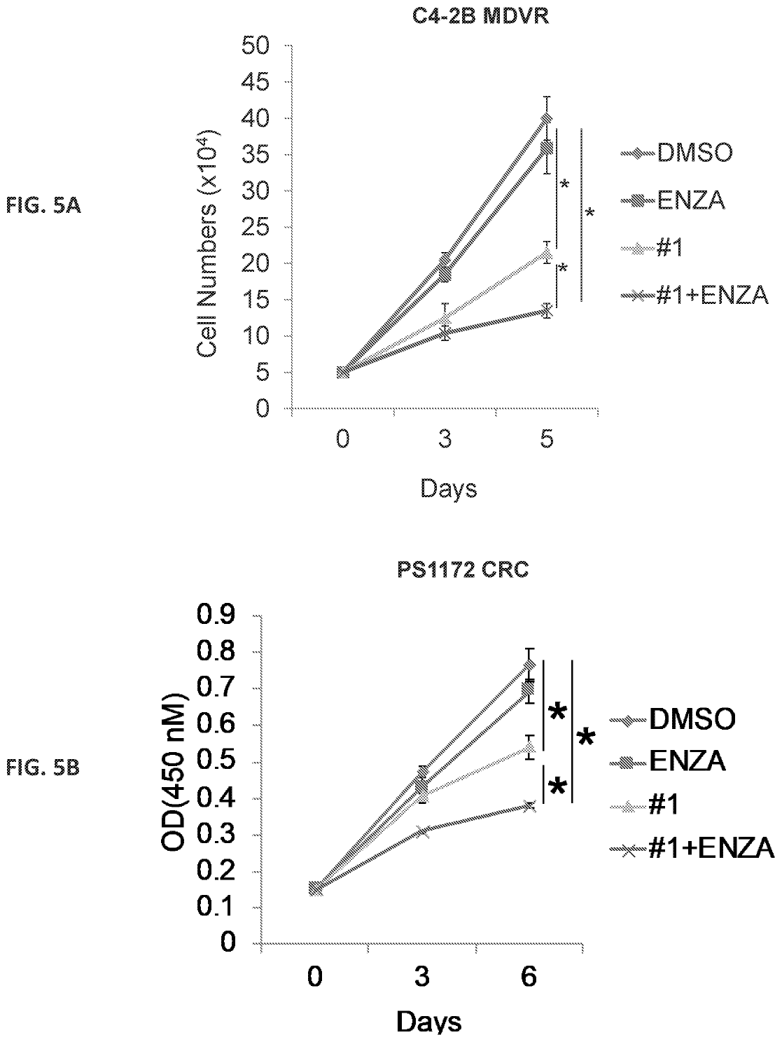

Knockdown of Wnt5a by siRNA specific to Wnt5a enhanced enzalutamide treatment in C4-2B MDVR and PS1172 CRC cells (FIGS. 5A-5B). Enzalutamide resistant C4-2B MDVR cells and PS1172 CRC cells were treated with either enzalutamide (20 μM) or Wnt5a siRNA (#1) or their combination (enza+#1) for 3 days and 5 days (C4-2B MDVR cells) or 3 days and 6 days (PS1172 CRC cells), and the cell numbers were determined. The results demonstrated that combination of Wnt5a siRNA and enzalutamide synergistically enhanced enzalutamide treatment.

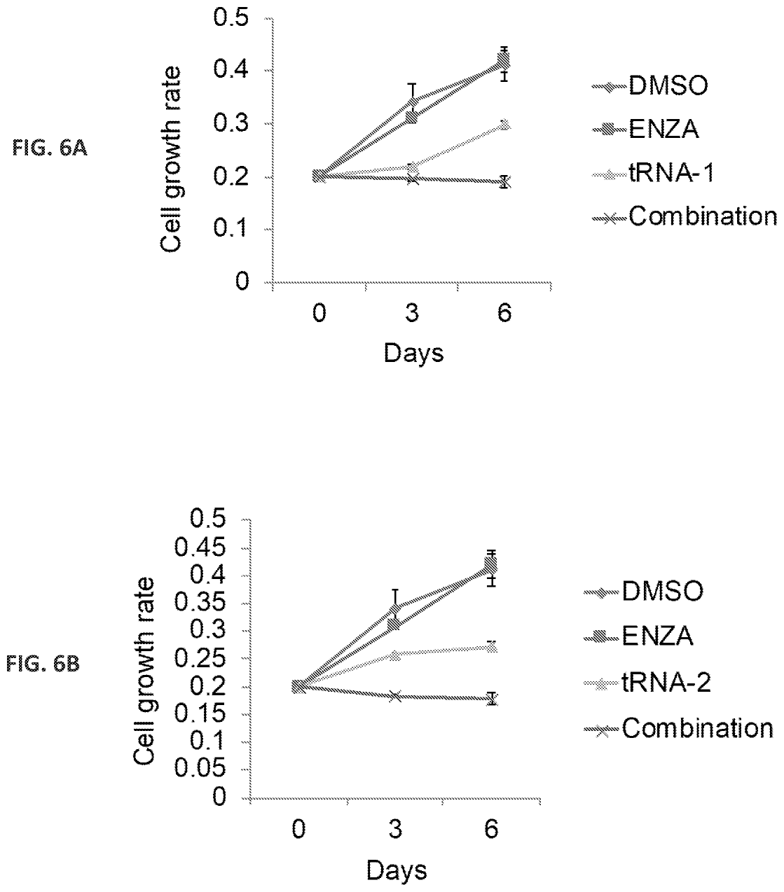

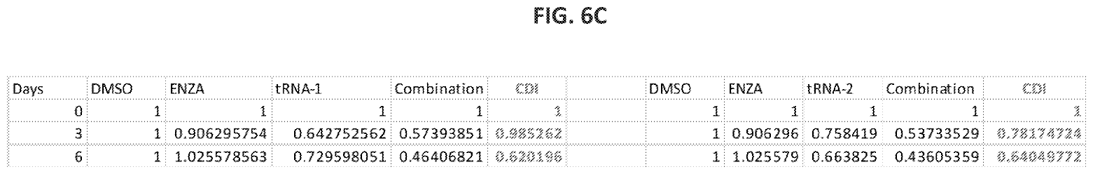

Knockdown of Wnt5a by tRNA-Wnt5a siRNA-1 (tRNA-1; see, e.g., FIG. 8A) and tRNA-Wnt5a siRNA-2 (tRNA-2; see, e.g., FIG. 8B) in C4-2B MDVR cells enhanced enzalutamide (ENZA) (FIGS. 6A-6B). Resistant C4-2B MDVR prostate cancer cells were treated with either enzalutamide (Enza, 20 uM) or tRNA-1 (10 nM) or tRNA-2 (10 nM) or their combination for 3 days and 6 days, and the cell numbers were determined. The co-efficient of drug interaction (CDI) is shown in FIG. 6C. A CDI<1 is considered synergism, and in particular a CDI<0.7 is considered significantly synergistic.

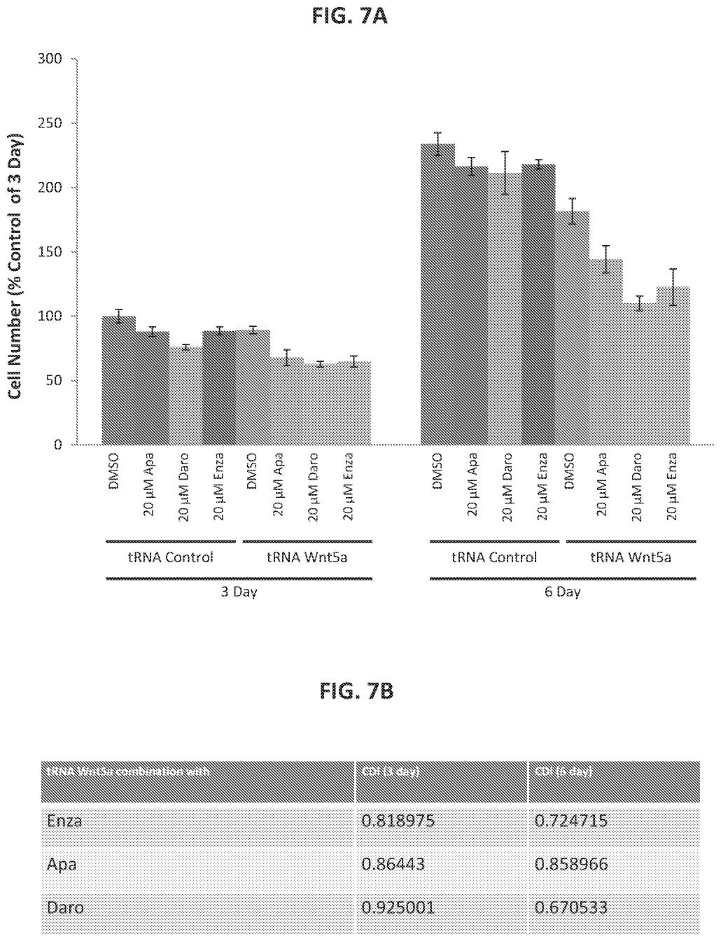

Knockdown of Wnt5a by tRNA-Wnt5a siRNA enhances anti-androgen (enzalutamide, apalutamide, darolutamide) treatments (FIG. 7A). Resistant C4-2B MDVR cells were treated with tRNA Wnt5a siRNA-2 and antiandrogens such as apalutamide (Apa), darolutamide (Daro), enzalutamide (enza) individually or their combination for 3 days and 6 days and the cell number was determined. The co-efficient of drug interaction (CDI) is shown in FIG. 7B. A CDI<1 is considered synergism, and in particular a CDI<0.7 is considered significantly synergistic.

### Example 3. Targeting WNT5A Enhances Enzalutamide Effects in LuCaP 35CR Organoids and PDX Model

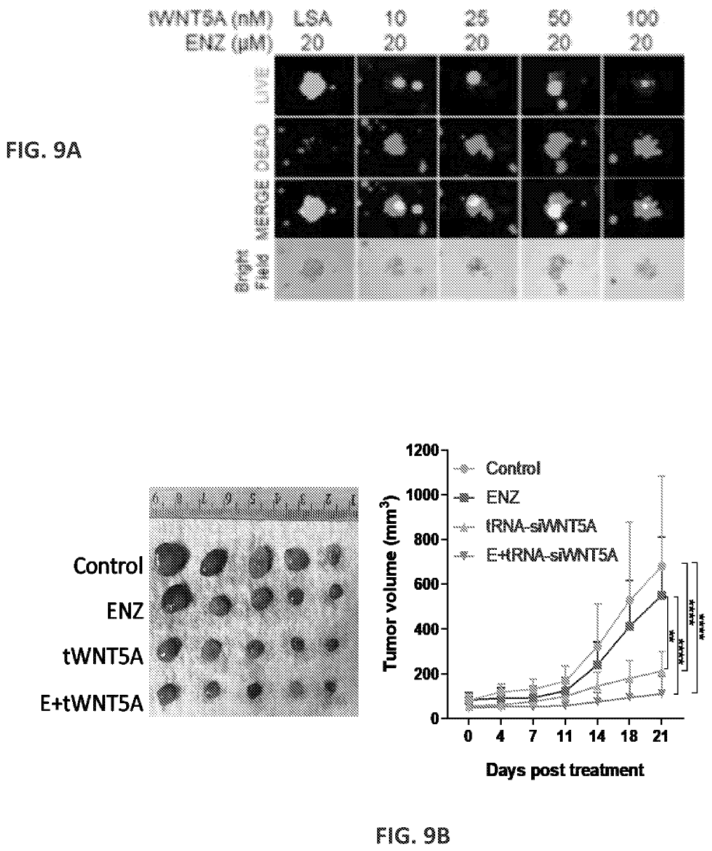

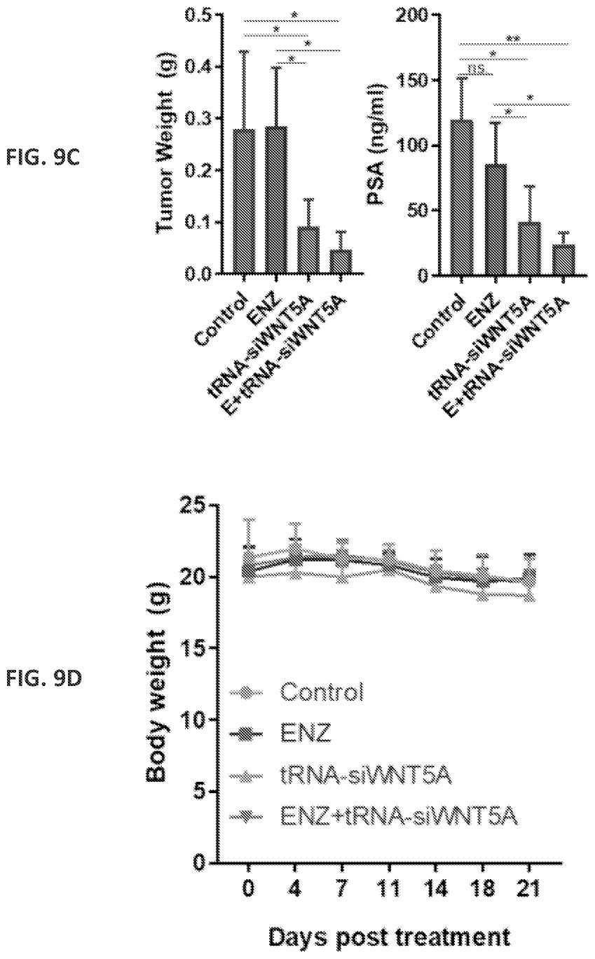

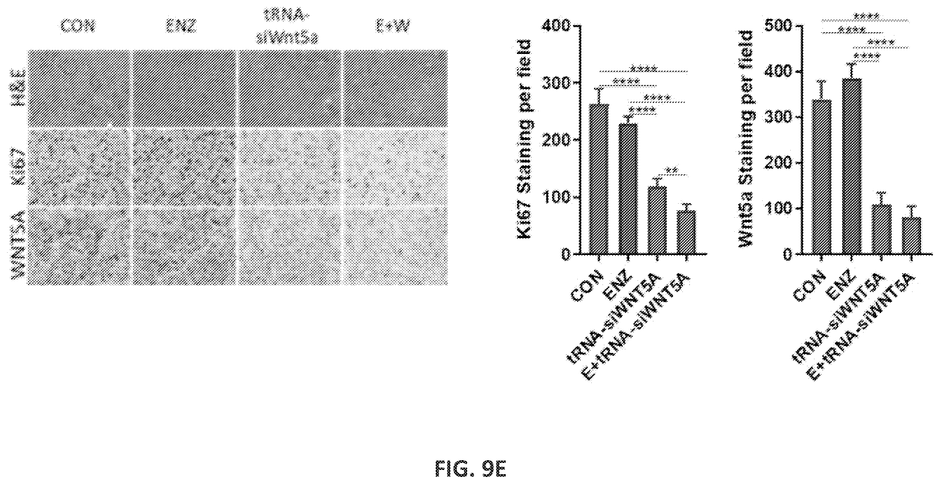

To examine if targeting Wnt5a enhances enzalutamide treatment in resistant prostate cancer, we examined the combinational effects of targeting Wnt5a and enzalutamide in an ex vivo model. Organoids derived from the LuCaP PDX model were established in an ex vivo 3D Matrigel format and treated with bioengineered BERA-Wnt5a siRNA. LuCaP 35CR organoids remained resistant to enzalutamide treatment, whereas combinational treatment with BERA-Wnt5a siRNA had robust anti-tumor effects in organoids (FIG. 9A). To further determine the anti-tumor effects of Wnt5a inhibition in vivo, we employed a LuCaP 35CR patient derived xenograft model, which was treated with bioengineered BERA-Wnt5a siRNA. LuCaP 35CR tumors were resistant to enzalutamide treatment (p>0.05), and single treatment of BERA-Wnt5a significantly inhibited tumor growth (p<0.05). A combination of BERA-Wnt5a with enzalutamide further suppressed tumor growth in LuCaP 35CR PDX tumors (p<0.05) (FIGS. 9B, 9C left). Enzalutamide treatment affected PSA expression without reaching significance (P>0.05), whereas the combinational treatment using BERA-Wnt5a siRNA with enzalutamide significantly reduced PSA (P<0.05) (FIG. 9C right). The treatment did not affect the mouse body weight (FIG. 9D). Immunohistochemical staining of Ki67 also allowed verification that cancer cell proliferation was significantly inhibited by Wnt5a inhibition alone, and that this effect was further enhanced by the combination treatment with enzalutamide (FIG. 9E). Collectively, our results indicates that inhibition via Wnt5a expression by bioengineered (BERA) siRNA reduces enzalutamide-resistant tumor growth, and that this effect can be further enhanced by combinational treatment with enzalutamide.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### EXEMPLARY EMBODIMENTS

Exemplary embodiments provided in accordance with the presently disclosed subject matter include, but are not limited to, the claims and the following embodiments:
1. A tRNA-pre-miRNA chimera for inhibiting the expression of Wnt5a in a cell, the chimera comprising:
   (i) a tRNA component comprising a first tRNA sequence at the 5′ terminus of the tRNA-pre-miRNA chimera, and a second tRNA sequence at the 3′ terminus of the tRNA-pre-miRNA chimera, wherein the first and second tRNA sequences hybridize to one another to form a tRNA structure; and
   (ii) a pre-miRNA sequence, located between the first and second tRNA sequences on the tRNA-pre-miRNA chimera, wherein the pre-miRNA sequence comprises an inserted heterologous Wnt5a-inhibiting RNA sequence.
2. The tRNA-pre-miRNA chimera of embodiment 1, wherein the heterologous Wnt5a-inhibiting RNA sequence is an siRNA or mature microRNA (mi-RNA).
3. The tRNA-pre-miRNA chimera of embodiment 1 or 2, wherein the pre-miRNA sequence is derived from miRNA-34a.
4. The tRNA-pre-miRNA chimera of any one of embodiments 1 to 3, wherein the pre-miRNA sequence is derived from a mammalian pre-miRNA.
5. The tRNA-pre-miRNA chimera of embodiment 4, wherein the mammalian pre-miRNA is a human pre-miRNA.
6. The tRNA-pre-miRNA chimera of any one of embodiments 1 to 5, wherein the first and/or second tRNA sequences are derived from a mammalian tRNA.
7. The tRNA-pre-miRNA chimera of embodiment 6, wherein the mammalian tRNA is a human tRNA.
8. The tRNA-pre-miRNA chimera of any one of embodiments 1 to 7, wherein the first and/or second tRNA sequences are derived from a tRNA coding for an amino acid selected from the group consisting of serine, leucine, glycine, glutamate, aspartate, glutamine, arginine, cysteine, lysine, methionine, asparagine, alanine, histidine, isoleucine, phenylalanine, proline, tryptophan, tyrosine, threonine, and valine.
9. The tRNA-pre-miRNA chimera of embodiment 8, wherein the first and/or second tRNA sequences are derived from a tRNA coding for leucine.
10. The tRNA-pre-miRNA chimera of any one of embodiments 1 to 9, wherein the pre-miRNA sequence comprises:
   (a) a first pre-miRNA-34a sequence;
   (b) a Wnt5a miRNA or siRNA sequence;
   (c) a second pre-miRNA-34a sequence;
   (d) a complementary Wnt5a miRNA or siRNA sequence; and
   (e) a third pre-miRNA-34a sequence;
   wherein the first and third pre-miRNA-34a sequences hybridize to one another to form a pre-miRNA structure adjacent to the tRNA structure;
   wherein the Wnt5a miRNA or siRNA sequence and the complementary Wnt5a miRNA or siRNA sequence hybridize to one another to form a double-stranded RNA segment adjacent to the pre-miRNA structure, on the opposite side of the pre-miRNA structure as the tRNA structure; and
   wherein the second pre-miRNA-34a sequence forms a stem-loop structure adjacent to the double-stranded RNA segment, on the opposite side of the double-stranded RNA segment as the pre-miRNA structure.
11. The tRNA-pre-miRNA chimera of any one of embodiments 1 to 10, wherein the heterologous Wnt5a-inhibiting RNA sequence is inserted at, abutted with, or operably linked to a dicer or RNAse cleavage site within the pre-miRNA sequence.
12. The tRNA-pre-miRNA chimera of any one of embodiment 1 to 11, wherein the first tRNA sequence comprises the sequence shown as SEQ ID NO:9 or SEQ ID NO:10.
13. The tRNA-pre-miRNA chimera of any one of embodiments 1 to 12, wherein the second tRNA sequence comprises the sequence shown as SEQ ID NO:11 or SEQ ID NO:12.
14. The tRNA-pre-miRNA chimera of any one of embodiments 10 to 13, wherein the first pre-miRNA-34a sequence comprises the sequence shown as SEQ ID NO:13 or SEQ ID NO:14.
15. The tRNA-pre-miRNA chimera of any one of embodiments 10 to 14, wherein the second pre-miRNA-34a sequence comprises the sequence shown as SEQ ID NO:15, SEQ ID NO:16, or SEQ ID NO:17.
16. The tRNA-pre-miRNA chimera of any one of embodiments 10 to 15, wherein the third pre-miRNA-34a sequence comprises the sequence shown as SEQ ID NO:18 or SEQ ID NO:19.
17. The tRNA-pre-miRNA chimera of any one of embodiments 10 to 16, wherein the Wnt5a siRNA sequence comprises the sequence shown as SEQ ID NO:3, SEQ ID NO:4, SEQ ID NO:5 , or SEQ ID NO:6.
18. The tRNA-pre-miRNA chimera of any one of embodiments 10 to 17, wherein the complementary Wnt5a siRNA sequence comprises the sequence shown as SEQ ID NO:7 or SEQ ID NO:8.
19. The tRNA-pre-miRNA chimera of any one of embodiments 10 to 18, wherein the tRNA-pre-miRNA chimera comprises the sequence shown as SEQ ID NO:20 or SEQ ID NO:21.
20. The tRNA-pre-miRNA chimera of any one of embodiments 10 to 19, wherein the tRNA-pre-miRNA chimera comprises the sequence shown as SEQ ID NO:22, wherein (N1) corresponds to the Wnt5a siRNA or miRNA sequence, of length n, and (N2) corresponds to the complementary Wnt5a siRNA or miRNA sequence, also of length n.
21. The tRNA-pre-miRNA chimera of any one of embodiments 1 to 20, wherein the introduction of the chimera into a mammalian cell results in the processing of the chimera and release of the heterologous Wnt5a-inhibiting RNA sequence in the cell.
22. The tRNA-pre-miRNA chimera of embodiment 21, wherein the mammalian cell expresses Wnt5a, and wherein the introduction of the chimera into the cell leads to a reduction in Wnt5a expression in the cell.
23. The tRNA-pre-miRNA chimera of embodiment 21 or 22, wherein the mammalian cell is a human cell.
24. The tRNA-pre-miRNA chimera of any one of embodiments 21 to 23, wherein the mammalian cell is a cancer cell.
25. The tRNA-pre-miRNA chimera of embodiment 24, wherein the cancer cell is a prostate cancer cell.
26. The tRNA-pre-miRNA chimera of embodiment 24 or 25, wherein the introduction of the chimera into the cancer cell inhibits the growth of the cell.
27. The tRNA-pre-miRNA chimera of any one of embodiments 24 to 26, wherein the cancer cell is resistant to an antiandrogen, and wherein the introduction of the tRNA-pre-miRNA chimera into the cell sensitizes the cell to the antiandrogen.
28. The tRNA-pre-miRNA chimera of any one of embodiments 24 to 27, wherein the tRNA-pre-miRNA chimera and the antiandrogen act synergistically to inhibit the growth of the cancer cell.
29. The tRNA-pre-miRNA chimera of embodiment 28, wherein the co-efficient drug interaction (CDI) of the tRNA-pre-miRNA chimera and the antiandrogen is less than about 0.95, 0.90, 0.85, 0.80, 0.75, or 0.70.
30. The tRNA-pre-miRNA chimera of any one of embodiments 27 to 29, wherein the antiandrogen is selected from the group consisting of enzalutamide, apalutamide, and darolutamide.
31. A composition comprising the tRNA-pre-miRNA chimera of any one of embodiments 1 to 30 and an antiandrogen.
32. The composition of embodiment 31, wherein the antiandrogen is selected from the group consisting of enzalutamide, apalutamide, and darolutamide.
33. The composition of embodiment 31 or 32, wherein the tRNA-pre-miRNA chimera and the antiandrogen act synergistically to inhibit the growth of a Wnt5a-expressing cancer cell.
34. The composition of embodiment 33, wherein the cancer cell is a prostate cancer cell.
35. The composition of embodiment 33 or 34, wherein the co-efficient of drug interaction (CDI) of the tRNA-pre-miRNA chimera and the antiandrogen is less than about 0.95, 0.85, 0.80, 0.75, or 0.70.
36. The composition of any one of embodiments 31 to 35, wherein the tRNA-pre-miRNA chimera is present in an amount effective to reduce or reverse resistance of a cancer cell to antiandrogen.
37. The composition of embodiment 36, wherein the cancer cell is a prostate cancer cell.
38. An expression cassette comprising a polynucleotide encoding the tRNA-pre-miRNA chimera of any one of embodiments 1 to 30, operably linked to a promoter.
39. A host cell comprising the expression cassette of embodiment 38.
40. The host cell of embodiment 39, wherein the host cell is a bacterial host cell.
41. The bacterial host cell of embodiment 40, wherein the host cell is E. coli.
42. A method of inhibiting the growth of a Wnt5a-expressing cancer cell, the method comprising contacting the cell with the tRNA-pre-miRNA chimera of any one of embodiments 1 to 30, or the composition of any one of embodiments 31 to 37.
43. The method of embodiment 42, wherein the tRNA-pre-miRNA chimera is processed in the cell, leading to the release of the heterologous Wnt5a-inhibiting RNA sequence in the cell.
44. The method of embodiment 42 or 43, wherein the tRNA-pre-miRNA chimera inhibits the expression of Wnt5a in the cell.
45. The method of any one of embodiments 42 to 44, wherein the cell is resistant to an antiandrogen, and wherein the method further comprises contacting the cell with antiandrogen.
46. The method of embodiment 45, wherein the tRNA-pre-miRNA chimera and antiandrogen act synergistically to inhibit the growth of the cancer cell.
47. The method of embodiment 46, wherein the co-efficient drug interaction (CDI) of the tRNA-pre-miRNA chimera and the antiandrogen is less than about 0.95, 0.90, 0.85, 0.75, or 0.70.
48. The method of any one of embodiments 45 to 47, wherein the antiandrogen is selected from the group consisting of enzalutamide, apalutamide, and darolutamide.
49. The method of any one of embodiments 42 to 48, wherein the cancer cell is a prostate cancer cell.
50. The method of any one of embodiments 42 to 49, wherein the cancer cell is a mammalian cell.
51. The method of embodiment 50, wherein the mammalian cell is a human cell.
52. The method of any one of embodiments 42 to 51, wherein the tRNA-pre-miRNA chimera is provided by culturing the host cell of any one of embodiments 39 to 41 under conditions conducive to the expression of the tRNA-pre-miRNA chimera, and purifying the tRNA-pre-miRNA chimera from the host cell.
53. A method of treating a subject with a Wnt5a-expressing cancer, the method comprising administering to the subject the tRNA-pre-miRNA chimera of any one of embodiments 1 to 30, or the composition of any one of embodiments 31 to 37.
54. The method of embodiment 53, wherein the cancer is resistant to an antiandrogen, and wherein the method further comprises administering the antiandrogen to the subject.
55. The method of embodiment 54, wherein the antiandrogen is selected from the group consisting of enzalutamide, apalutamide, and darolutamide.
56. The method of any one of embodiments 53 to 55, wherein the method results in a decrease in the expression of Wnt5a in one or more Wnt5a-expressing cancer cells in the subject.
57. The method of any one of embodiments 53 to 56, wherein the method results in a decrease in tumor growth in the subject.
58. The method of any one of embodiments 53 to 57, wherein the cancer is prostate cancer.
59. The method of embodiment 58, wherein the method results in a decrease in serum PSA levels in the subject.
60. The method of any one of embodiments 53 to 59, wherein the method does not alter the body weight of the subject.
61. The method of any one of embodiments 53 to 60, wherein the subject is a human.
62. The method of any one of embodiments 53 to 61, wherein the tRNA-pre-miRNA chimera is administered to the subject through intravenous injection.
63. The method of any one of embodiments 53 to 62, wherein the tRNA-pre-miRNA chimera is packaged with lipopolyplex prior to administration to the subject.

INFORMAL SEQUENCE LISTING

SEQ ID NO: 1
Wnt5a siRNA-1:
5′-ACAAACUGGUCCACGAUCUCCGUGC-3′
SEQ ID NO: 2
Wnt5a siRNA-2:
5′-CUAGGAAGAACUUGGAAGACAUUGC-3′
SEQ ID NO: 3
tRNA-Wnt5a siRNA-1 (siRNA sequence within tRNA-pre-miRNA chimera #1):
ACAAACUGGUCCACGAUCUCCG
SEQ ID NO: 4
RNA-Wnt5a siRNA-2 (siRNA sequence within tRNA-pre-miRNA chimera #2):
CUAGGAAGAACUUGGAAGACAU
SEQ ID NO: 5
Alternative tRNA-Wnt5a siRNA (siRNA sequence within tRNA-pre-miRNA chimera):
5′-ACAAACUGGUCCACGAUCUCCGUGC-3′
SEQ ID NO: 6
Alternative tRNA-Wnt5a siRNA (siRNA sequence within tRNA-pre-miRNA chimera):
5′-CUAGGAAGAACUUGGAAGACAUUGC-3′
SEQ ID NO: 7
tRNA-Wnt5a siRNA-complementary sequence #1 (sequence complementary to siRNA within
tRNA-pre-miRNA chimera #1):
GGAGAUCGGGAUCCAGUUUGCU
SEQ ID NO: 8
tRNA-Wnt5a siRNA-complementary sequence #2 (sequence complementary to siRNA within
tRNA-pre-miRNA chimera #2):
UGUCUUCCGGUCUUUUCCUACU
SEQ ID NO: 9
5′ tRNA sequence (from htRNA^{Leu})(also provided is the same sequence in which each T is
replaced by U):
TTCTCAACATAAAAAACTTTGTGTAATACTTGTAACGCTGAATTC
SEQ ID NO: 10
Alternative 5′ tRNA sequence:
GGCUACGUAGCUCAGUUGGUUAGAGCAGCGGCCGAGUAAUUUACGUCGAC
SEQ ID NO: 11
3′ tRNA sequence (from htRNA^{Leu})(also provided is the same sequence in which each T is
replaced by U):
CTGCAGATCCTTAGCGAAAGCTAAGGATTTTTTTT
SEQ ID NO: 12
Alternative 3′ tRNA sequence:
GACGUCGAUGGUUGCGGCCGCGGGUCACAGGUUCGAAUCCCGUCGUAGCCACCA
SEQ ID NO: 13
5′ pre-miRNA-34a sequence (also provided is the same sequence in which each T is replaced by
U):
ACCAGGATGGCCGAGTGGTTAAGGCGTTGGACT
SEQ ID NO: 14
Alternative 5′ pre-miRNA-34a sequence:
CCGUGGACCGGCCAGCUGUGAGUGUUUCUU
SEQ ID NO: 15
Central pre-miRNA-34a sequence (also provided is the same sequence in which each T is
replaced by U):
TGTGAGCAATAGTAAGGAAT
SEQ ID NO: 16
Central pre-miRNA-34a sequence (also provided is the same sequence in which each T is
replaced by U):
TGTGAGCAATAGTAAGGAAG
SEQ ID NO: 17
Alternative central pre-miRNA-34a sequence:
UGUGAGCAAUAGUAAGGAAG
SEQ ID NO: 18
3′ pre-miRNA-34a sequence (also provided is the same sequence in which each T is replaced by
U):
GATCCAATGGACATATGTCCGCGTGGGTTCGAACCCCACTCCTGGTACCA
SEQ ID NO: 19
Alternative 3′ pre-miRNA-34a sequence:
AGAAGUGCUGCACGUUGUGGGGCCCAAGAGGGAA
SEQ ID NO: 20
htRNA^{Leu}_pre-mir34a/Wnt5a-siRNA#1. This chimera uses a humanized carrier (using human
tRNA) and provides high expression levels and overall yield. Red and green are the siRNA and
complementary sequences; underlined is hsa-pre-miR-34a, and the rest is htRNA^{Leu} in which the
codon sequence has been replaced with hsa-pre-miR-34a (also provided is the same sequence in
which each T is replaced by U).
TTCTCAACATAAAAAACTTTGTGTAATACTTGTAACGCTGAATTCACCAGGATGGCC
GAGTGGTTAAGGCGTTGGACTGGCCAGCTGTGAGTGTTTCTTACAAACUGGUCCACG
AUCUCCGTGTGAGCAATAGTAAGGAAUGGAGAUCGGGAUCCAGUUUGCUAGAAGT
GCTGCACGTTGTTGGCCCGATCCAATGGACATATGTCCGCGTGGGTTCGAACCCCAC
TCCTGGTACCACTGCAGATCCTTAGCGAAAGCTAAGGATTTTTTTT
SEQ ID NO: 21
htRNA^{Leu}_pre-mir34a/Wnt5a-siRNA#2. This chimera uses a humanized carrier (using human
tRNA) and provides high expression levels and overall yield. Red and green are the siRNA and
complementary sequences; underlined is hsa-pre-miR-34a, and the rest is htRNA^{Leu} in which the
codon sequence has been replaced with hsa-pre-miR-34a (also provided is the same sequence in
which each T is replaced by U).
TTCTCAACATAAAAAACTTTGTGTAATACTTGTAACGCTGAATTCACCAGGATGGCC
GAGTGGTTAAGGCGTTGGACTGGCCAGCTGTGAGTGTTTCTTCUAGGAAGAACUUG
GAAGACAUTGTGAGCAATAGTAAGGAAGUGUCUUCCGGUCUUUUCCUACUAGAAG
TGCTGCACGTTGTTGGCCCGATCCAATGGACATATGTCCGCGTGGGTTCGAACCCCA
CTCCTGGTACCACTGCAGATCCTTAGCGAAAGCTAAGGATTTTTTTT
SEQ ID NO: 22
Alternative Wnt5a tRNA-miRNA chimera (underlined sequences = tRNA; N1 = Wnt5a miRNA or
siRNA sequence, N2 = sequence complementary to Wnt5a miRNA or siRNA sequence, or vice
versa):
GGCUACGUAGCUCAGUUGGUUAGAGCAGCGGCCGAGUAAUUUACGUCGACCCGU
GGACCGGCCAGCUGUGAGUGUUUCUU(N1)ₙUGUGAGCAAUAGUAAGGAAG(N2)ₙAG
AAGUGCUGCACGUUGUGGGGCCCAAGAGGGAAGACGUCGAUGGUUGCGGCCGCG
GGUCACAGGUUCGAAUCCCGUCGUAGCCACCA

What is claimed is:

1. A tRNA-pre-miRNA chimera for inhibiting the expression of Wnt5a in a cell, the chimera comprising:
(i) a tRNA component comprising a first tRNA sequence at the 5′ terminus of the tRNA-pre-miRNA chimera, and a second tRNA sequence at the 3′ terminus of the tRNA-pre-miRNA chimera, wherein the first and second tRNA sequences hybridize to one another to form a tRNA structure; and
(ii) a pre-miRNA sequence, located between the first and second tRNA sequences on the tRNA-pre-miRNA chimera, wherein the pre-miRNA sequence comprises an inserted heterologous Wnt5a-inhibiting RNA sequence.

2. The tRNA-pre-miRNA chimera of claim 1, wherein the heterologous Wnt5a-inhibiting RNA sequence is an siRNA or mature microRNA (mi-RNA).

3. The tRNA-pre-miRNA chimera of claim 1, wherein the pre-miRNA sequence is derived from miRNA-34a.

4. The tRNA-pre-miRNA chimera of claim 1, wherein the pre-miRNA sequence is derived from a mammalian pre-miRNA.

5. The tRNA-pre-miRNA chimera of claim 4, wherein the mammalian pre-miRNA is a human pre-miRNA.

6. The tRNA-pre-miRNA chimera of claim 1, wherein the first and/or second tRNA sequences are derived from a mammalian tRNA.

7. The tRNA-pre-miRNA chimera of claim 6, wherein the mammalian tRNA is a human tRNA.

8. The tRNA-pre-miRNA chimera of claim 1, wherein the first and/or second tRNA sequences are derived from a tRNA coding for an amino acid selected from the group consisting of serine, leucine, glycine, glutamate, aspartate, glutamine, arginine, cysteine, lysine, methionine, asparagine, alanine, histidine, isoleucine, phenylalanine, proline, tryptophan, tyrosine, threonine, and valine.

9. The tRNA-pre-miRNA chimera of claim 8, wherein the first and/or second tRNA sequences are derived from a tRNA coding for leucine.

10. The tRNA-pre-miRNA chimera of claim 1, wherein the pre-miRNA sequence comprises:
(a) a first pre-miRNA-34a sequence;
(b) a Wnt5a miRNA or siRNA sequence;
(c) a second pre-miRNA-34a sequence;
(d) a complementary Wnt5a miRNA or siRNA sequence; and
(e) a third pre-miRNA-34a sequence;
wherein the first and third pre-miRNA-34a sequences hybridize to one another to form a pre-miRNA structure adjacent to the tRNA structure;
wherein the Wnt5a miRNA or siRNA sequence and the complementary Wnt5a miRNA or siRNA sequence hybridize to one another to form a double-stranded RNA segment adjacent to the pre-miRNA structure, on the opposite side of the pre-miRNA structure as the tRNA structure; and
wherein the second pre-miRNA-34a sequence forms a stem-loop structure adjacent to the double-stranded RNA segment, on the opposite side of the double-stranded RNA segment as the pre-miRNA structure.

11. The tRNA-pre-miRNA chimera of claim 1, wherein the heterologous Wnt5a-inhibiting RNA sequence is inserted at, abutted with, or operably linked to a dicer or RNAse cleavage site within the pre-miRNA sequence.

12. The tRNA-pre-miRNA chimera of claim 1, wherein the first tRNA sequence comprises the sequence shown as SEQ ID NO:9 or SEQ ID NO:10.

13. The tRNA-pre-miRNA chimera of claim 1, wherein the second tRNA sequence comprises the sequence shown as SEQ ID NO:11 or SEQ ID NO:12.

14. The tRNA-pre-miRNA chimera of claim 10, wherein the first pre-miRNA-34a sequence comprises the sequence shown as SEQ ID NO:13 or SEQ ID NO:14.

15. The tRNA-pre-miRNA chimera of claim 10, wherein the second pre-miRNA-34a sequence comprises the sequence shown as SEQ ID NO:15, SEQ ID NO:16, or SEQ ID NO:17.

16. The tRNA-pre-miRNA chimera of claim 10, wherein the third pre-miRNA-34a sequence comprises the sequence shown as SEQ ID NO:18 or SEQ ID NO:19.

17. The tRNA-pre-miRNA chimera of claim 10, wherein the Wnt5a siRNA sequence comprises the sequence shown as SEQ ID NO:3, SEQ ID NO:4, SEQ ID NO:5 , or SEQ ID NO:6.

18. The tRNA-pre-miRNA chimera of claim 10, wherein the complementary Wnt5a siRNA sequence comprises the sequence shown as SEQ ID NO:7 or SEQ ID NO:8.

19. The tRNA-pre-miRNA chimera of claim 10, wherein the tRNA-pre-miRNA chimera comprises the sequence shown as SEQ ID NO:20 or SEQ ID NO:21.

20. The tRNA-pre-miRNA chimera of claim 10, wherein the tRNA-pre-miRNA chimera comprises the sequence shown as SEQ ID NO:22, wherein (N1) corresponds to the Wnt5a siRNA or miRNA sequence, of length n, and (N2) corresponds to the complementary Wnt5a siRNA or miRNA sequence, also of length n.

21. The tRNA-pre-miRNA chimera of claim 1, wherein the introduction of the chimera into a mammalian cell results in the processing of the chimera and release of the heterologous Wnt5a-inhibiting RNA sequence in the cell.

22. The tRNA-pre-miRNA chimera of claim 21, wherein the mammalian cell expresses Wnt5a, and wherein the introduction of the chimera into the cell leads to a reduction in Wnt5a expression in the cell.

23. The tRNA-pre-miRNA chimera of claim 21, wherein the mammalian cell is a human cell.

24. The tRNA-pre-miRNA chimera of claim 21, wherein the mammalian cell is a cancer cell.

25. The tRNA-pre-miRNA chimera of claim 24, wherein the cancer cell is a prostate cancer cell.

26. The tRNA-pre-miRNA chimera of claim 24, wherein the introduction of the chimera into the cancer cell inhibits the growth of the cell.

27. The tRNA-pre-miRNA chimera of claim 24, wherein the cancer cell is resistant to an antiandrogen, and wherein the introduction of the tRNA-pre-miRNA chimera into the cell sensitizes the cell to the antiandrogen.

28. The tRNA-pre-miRNA chimera of claim 24, wherein the tRNA-pre-miRNA chimera and the antiandrogen act synergistically to inhibit the growth of the cancer cell.

29. The tRNA-pre-miRNA chimera of claim 28, wherein the co-efficient drug interaction (CDI) of the tRNA-pre-miRNA chimera and the antiandrogen is less than about 0.95, 0.90, 0.85, 0.80, 0.75, or 0.70.

30. The tRNA-pre-miRNA chimera of claim 27, wherein the antiandrogen is selected from the group consisting of enzalutamide, apalutamide, and darolutamide.

31. A composition comprising the tRNA-pre-miRNA chimera of claim 1 and an antiandrogen.

32. The composition of claim 31, wherein the antiandrogen is selected from the group consisting of enzalutamide, apalutamide, and darolutamide.

33. The composition of claim 31, wherein the tRNA-pre-miRNA chimera and the antiandrogen act synergistically to inhibit the growth of a Wnt5a-expressing cancer cell.

34. The composition of claim 33, wherein the cancer cell is a prostate cancer cell.

35. The composition of claim 33, wherein the co-efficient of drug interaction (CDI) of the tRNA-pre-miRNA chimera and the antiandrogen is less than about 0.95, 0.90, 0.85, 0.80, 0.75, or 0.70.

36. The composition of claim 31, wherein the tRNA-pre-miRNA chimera is present in an amount effective to reduce or reverse resistance of a cancer cell to antiandrogen.

37. The composition of claim 36, wherein the cancer cell is a prostate cancer cell.

38. An expression cassette comprising a polynucleotide encoding the tRNA-pre-miRNA chimera of claim 1, operably linked to a promoter.

39. A host cell comprising the expression cassette of claim 38.

40. The host cell of claim 39, wherein the host cell is a bacterial host cell.

41. The bacterial host cell of claim 40, wherein the host cell is E. coli.

42. A method of inhibiting the growth of a Wnt5a-expressing cancer cell, the method comprising contacting the cell with the tRNA-pre-miRNA chimera of claim 1.

43. The method of claim 42, wherein the tRNA-pre-miRNA chimera is processed in the cell, leading to the release of the heterologous Wnt5a-inhibiting RNA sequence in the cell.

44. The method of claim 42, wherein the tRNA-pre-miRNA chimera inhibits the expression of Wnt5a in the cell.

45. The method of claim 42, wherein the cell is resistant to an antiandrogen, and wherein the method further comprises contacting the cell with antiandrogen.

46. The method of claim 45, wherein the tRNA-pre-miRNA chimera and antiandrogen act synergistically to inhibit the growth of the cancer cell.

47. The method of claim 46, wherein the co-efficient drug interaction (CDI) of the tRNA-pre-miRNA chimera and the antiandrogen is less than about 0.95, 0.90, 0.85, 0.80, 0.75, or 0.70.

48. The method of claim 45, wherein the antiandrogen is selected from the group consisting of enzalutamide, apalutamide, and darolutamide.

49. The method of claim 42, wherein the cancer cell is a prostate cancer cell.

50. The method of claim 42, wherein the cancer cell is a mammalian cell.

51. The method of claim 50, wherein the mammalian cell is a human cell.

52. The method of claim 42, wherein the tRNA-pre-miRNA chimera is provided by culturing the host cell of claim 39 under conditions conducive to the expression of the tRNA-pre-miRNA chimera, and purifying the tRNA-pre-miRNA chimera from the host cell.

53. A method of treating a subject with a Wnt5a-expressing cancer, the method comprising administering to the subject the tRNA-pre-miRNA chimera of claim 1.

54. The method of claim 53, wherein the cancer is resistant to an antiandrogen, and wherein the method further comprises administering the antiandrogen to the subject.

55. The method of claim 54, wherein the antiandrogen is selected from the group consisting of enzalutamide, apalutamide, and darolutamide.

56. The method of claim 53, wherein the method results in a decrease in the expression of Wnt5a in one or more Wnt5a-expressing cancer cells in the subject.

57. The method of claim 53, wherein the method results in a decrease in tumor growth in the subject.

58. The method of claim 53, wherein the cancer is prostate cancer.

59. The method of claim 58, wherein the method results in a decrease in serum PSA levels in the subject.

60. The method of claim 53, wherein the method does not alter the body weight of the subject.

61. The method of claim 53, wherein the subject is a human.

62. The method of claim 53, wherein the tRNA-pre-miRNA chimera is administered to the subject through intravenous injection.

63. The method of claim 53, wherein the tRNA-pre-miRNA chimera is packaged with lipopolyplex prior to administration to the subject.


SEQUENCE LISTING (24 sequences)

SEQ ID NO: 1 - RNA, 25 nt, Artificial Sequence
  Description of Artificial Sequence Synthetic oligonucleotide
acaaacugguccacgaucuccgugc

SEQ ID NO: 2 - RNA, 25 nt, Artificial Sequence
  Description of Artificial Sequence Synthetic oligonucleotide
cuaggaagaacuuggaagacauugc

SEQ ID NO: 3 - RNA, 22 nt, Artificial Sequence
  Description of Artificial Sequence Synthetic oligonucleotide
acaaacugguccacgaucuccg

SEQ ID NO: 4 - RNA, 22 nt, Artificial Sequence
  Description of Artificial Sequence Synthetic oligonucleotide
cuaggaagaacuuggaagacau

SEQ ID NO: 5 - RNA, 25 nt, Artificial Sequence
  Description of Artificial Sequence Synthetic oligonucleotide
acaaacugguccacgaucuccgugc

SEQ ID NO: 6 - RNA, 25 nt, Artificial Sequence
  Description of Artificial Sequence Synthetic oligonucleotide
cuaggaagaacuuggaagacauugc

SEQ ID NO: 7 - RNA, 22 nt, Artificial Sequence
  Description of Artificial Sequence Synthetic oligonucleotide
ggagaucgggauccaguuugcu

SEQ ID NO: 8 - RNA, 22 nt, Artificial Sequence
  Description of Artificial Sequence Synthetic oligonucleotide
ugucuuccggucuuuuccuacu

SEQ ID NO: 9 - DNA, 45 bp, Artificial Sequence
  Description of Artificial Sequence Synthetic oligonucleotide
  modified_base (1)..(2): t or u
  modified_base (4)..(4): t or u
  modified_base (10)..(10): t or u
  modified_base (18)..(20): t or u
  modified_base (22)..(22): t or u
  modified_base (24)..(24): t or u
  modified_base (27)..(27): t or u
  modified_base (30)..(31): t or u
  modified_base (33)..(33): t or u
  modified_base (39)..(39): t or u
  modified_base (43)..(44): t or u
  See specification as filed for detailed description of substitutions and preferred embodiments
nncncaacanaaaaaacnnngngnaanacnngnaacgcngaannc

SEQ ID NO: 10 - RNA, 50 nt, Artificial Sequence
  Description of Artificial Sequence Synthetic oligonucleotide
ggcuacguagcucaguugguuagagcagcggccgaguaauuuacgucgac

SEQ ID NO: 11 - DNA, 35 bp, Artificial Sequence
  Description of Artificial Sequence Synthetic oligonucleotide
  modified_base (2)..(2): t or u
  modified_base (8)..(8): t or u
  modified_base (11)..(12): t or u
  modified_base (22)..(22): t or u
  modified_base (28)..(35): t or u
  See specification as filed for detailed description of substitutions and preferred embodiments
cngcaganccnnagcgaaagcnaaggannnnnnnn

SEQ ID NO: 12 - RNA, 54 nt, Artificial Sequence
  Description of Artificial Sequence Synthetic oligonucleotide
gacgucgaugguugcggccgcgggucacagguucgaaucccgucguagccacca

SEQ ID NO: 13 - DNA, 33 bp, Artificial Sequence
  Description of Artificial Sequence Synthetic oligonucleotide
  modified_base (8)..(8): t or u
  modified_base (16)..(16): t or u
  modified_base (19)..(20): t or u
  modified_base (27)..(28): t or u
  modified_base (33)..(33): t or u
  See specification as filed for detailed description of substitutions and preferred embodiments
accagganggccgagnggnnaaggcgnnggacn

SEQ ID NO: 14 - RNA, 30 nt, Artificial Sequence
  Description of Artificial Sequence Synthetic oligonucleotide
ccguggaccggccagcugugaguguuucuu

SEQ ID NO: 15 - DNA, 20 bp, Artificial Sequence
  Description of Artificial Sequence Synthetic oligonucleotide
  modified_base (1)..(1): t or u
  modified_base (3)..(3): t or u
  modified_base (10)..(10): t or u
  modified_base (13)..(13): t or u
  modified_base (20)..(20): t or u
  See specification as filed for detailed description of substitutions and preferred embodiments
ngngagcaanagnaaggaan

SEQ ID NO: 16 - DNA, 20 bp, Artificial Sequence
  Description of Artificial Sequence Synthetic oligonucleotide
  modified_base (1)..(1): t or u
  modified_base (3)..(3): t or u
  modified_base (10)..(10): t or u
  modified_base (13)..(13): t or u
  See specification as filed for detailed description of substitutions and preferred embodiments
ngngagcaanagnaaggaag

SEQ ID NO: 17 - RNA, 20 nt, Artificial Sequence
  Description of Artificial Sequence Synthetic oligonucleotide
ugugagcaauaguaaggaag

SEQ ID NO: 18 - DNA, 50 bp, Artificial Sequence
  Description of Artificial Sequence Synthetic oligonucleotide
  modified_base (3)..(3): t or u
  modified_base (8)..(8): t or u
  modified_base (14)..(14): t or u
  modified_base (16)..(16): t or u
  modified_base (18)..(18): t or u
  modified_base (24)..(24): t or u
  modified_base (28)..(29): t or u
  modified_base (40)..(40): t or u
  modified_base (43)..(43): t or u
  modified_base (46)..(46): t or u
  See specification as filed for detailed description of substitutions and preferred embodiments
ganccaanggacanangnccgcgngggnncgaaccccacnccnggnacca

SEQ ID NO: 19 - RNA, 34 nt, Artificial Sequence
  Description of Artificial Sequence Synthetic oligonucleotide
agaagugcugcacguuguggggcccaagagggaa

SEQ ID NO: 20 - DNA, 272 bp, Artificial Sequence
  Description of Artificial Sequence Synthetic oligonucleotide
  Description of Combined DNA/RNA Molecule Synthetic oligonucleotide
  modified_base (1)..(2): t or u
  modified_base (4)..(4): t or u
  modified_base (10)..(10): t or u
  modified_base (18)..(20): t or u
  modified_base (22)..(22): t or u
  modified_base (24)..(24): t or u
  modified_base (27)..(27): t or u
  modified_base (30)..(31): t or u
  modified_base (33)..(33): t or u
  modified_base (39)..(39): t or u
  modified_base (43)..(44): t or u
  modified_base (53)..(53): t or u
  modified_base (61)..(61): t or u
  modified_base (64)..(65): t or u
  modified_base (72)..(73): t or u
  modified_base (78)..(78): t or u
  modified_base (86)..(86): t or u
  modified_base (88)..(88): t or u
  modified_base (92)..(92): t or u
  modified_base (94)..(96): t or u
  modified_base (98)..(99): t or u
  modified_base (122)..(122): t or u
  modified_base (124)..(124): t or u
  modified_base (131)..(131): t or u
  modified_base (134)..(134): t or u
  modified_base (169)..(169): t or u
  modified_base (172)..(172): t or u
  modified_base (178)..(179): t or u
  modified_base (181)..(182): t or u
  modified_base (190)..(190): t or u
  modified_base (195)..(195): t or u
  modified_base (201)..(201): t or u
  modified_base (203)..(203): t or u
  modified_base (205)..(205): t or u
  modified_base (211)..(211): t or u
  modified_base (215)..(216): t or u
  modified_base (227)..(227): t or u
  modified_base (230)..(230): t or u
  modified_base (233)..(233): t or u
  modified_base (239)..(239): t or u
  modified_base (245)..(245): t or u
  modified_base (248)..(249): t or u
  modified_base (259)..(259): t or u
  modified_base (265)..(272): t or u
  See specification as filed for detailed description of substitutions and preferred embodiments
nncncaacanaaaaaacnnngngnaanacnngnaacgcngaanncaccagganggccgagnggnnaaggcgnnggacnggccagcngngagngnnncnnacaaacugguccacgaucuccgngngagcaanagnaaggaauggagaucgggauccaguuugcuagaagngcngcacgnngnnggcccganccaanggacanangnccgcgngggnncgaaccccacnccnggnaccacngcaganccnnagcgaaagcnaaggannnnnnnn

SEQ ID NO: 21 - DNA, 272 bp, Artificial Sequence
  Description of Artificial Sequence Synthetic oligonucleotide
  Description of Combined DNA/RNA Molecule Synthetic oligonucleotide
  modified_base (1)..(2): t or u
  modified_base (4)..(4): t or u
  modified_base (10)..(10): t or u
  modified_base (18)..(20): t or u
  modified_base (22)..(22): t or u
  modified_base (24)..(24): t or u
  modified_base (27)..(27): t or u
  modified_base (30)..(31): t or u
  modified_base (33)..(33): t or u
  modified_base (39)..(39): t or u
  modified_base (43)..(44): t or u
  modified_base (53)..(53): t or u
  modified_base (61)..(61): t or u
  modified_base (64)..(65): t or u
  modified_base (72)..(73): t or u
  modified_base (78)..(78): t or u
  modified_base (86)..(86): t or u
  modified_base (88)..(88): t or u
  modified_base (92)..(92): t or u
  modified_base (94)..(96): t or u
  modified_base (98)..(99): t or u
  modified_base (122)..(122): t or u
  modified_base (124)..(124): t or u
  modified_base (131)..(131): t or u
  modified_base (134)..(134): t or u
  modified_base (169)..(169): t or u
  modified_base (172)..(172): t or u
  modified_base (178)..(179): t or u
  modified_base (181)..(182): t or u
  modified_base (190)..(190): t or u
  modified_base (195)..(195): t or u
  modified_base (201)..(201): t or u
  modified_base (203)..(203): t or u
  modified_base (205)..(205): t or u
  modified_base (211)..(211): t or u
  modified_base (215)..(216): t or u
  modified_base (227)..(227): t or u
  modified_base (230)..(230): t or u
  modified_base (233)..(233): t or u
  modified_base (239)..(239): t or u
  modified_base (245)..(245): t or u
  modified_base (248)..(249): t or u
  modified_base (259)..(259): t or u
  modified_base (265)..(272): t or u
  See specification as filed for detailed description of substitutions and preferred embodiments
nncncaacanaaaaaacnnngngnaanacnngnaacgcngaanncaccagganggccgagnggnnaaggcgnnggacnggccagcngngagngnnncnncuaggaagaacuuggaagacaungngagcaanagnaaggaagugucuuccggucuuuuccuacuagaagngcngcacgnngnnggcccganccaanggacanangnccgcgngggnncgaaccccacnccnggnaccacngcaganccnnagcgaaagcnaaggannnnnnnn

SEQ ID NO: 22 - RNA, 190 nt, Artificial Sequence
  Description of Artificial Sequence Synthetic polynucleotide
  modified_base (81)..(81): Wnt5a miRNA or siRNA sequence of length n
  modified_base (102)..(102): Sequence complementary to Wnt5a miRNA or siRNA sequence of length n
  See specification as filed for detailed description of substitutions and preferred embodiments
ggcuacguagcucaguugguuagagcagcggccgaguaauuuacgucgacccguggaccggccagcugugaguguuucuunugugagcaauaguaaggaagnagaagugcugcacguuguggggcccaagagggaagacgucgaugguugcggccgcgggucacagguucgaaucccgucguagccacca

SEQ ID NO: 23 - DNA, 272 bp, Artificial Sequence
  Description of Artificial Sequence Synthetic oligonucleotide
  Description of Combined DNA/RNA Molecule Synthetic oligonucleotide
ttctcaacataaaaaactttgtgtaatacttgtaacgctgaattcaccaggatggccgagtggttaaggcgttggactggccagctgtgagtgtttcttacaaacugguccacgaucuccgtgtgagcaatagtaaggaauggagaucgggauccaguuugcuagaagtgctgcacgttgttggcccgatccaatggacatatgtccgcgtgggttcgaaccccactcctggtaccactgcagatccttagcgaaagctaaggatttttttt

SEQ ID NO: 24 - DNA, 272 bp, Artificial Sequence
  Description of Artificial Sequence Synthetic oligonucleotide
  Description of Combined DNA/RNA Molecule Synthetic oligonucleotide
ttctcaacataaaaaactttgtgtaatacttgtaacgctgaattcaccaggatggccgagtggttaaggcgttggactggccagctgtgagtgtttcttcuaggaagaacuuggaagacautgtgagcaatagtaaggaagugucuuccggucuuuuccuacuagaagtgctgcacgttgttggcccgatccaatggacatatgtccgcgtgggttcgaaccccactcctggtaccactgcagatccttagcgaaagctaaggatttttttt